(19) United States
(12) Patent Application Publication
(10) Pub. No.: US 2019/0040083 A1
(43) Pub. Date: Feb. 7, 2019

(54) ANTIBACTERIAL COMPOUNDS

(71) Applicant: Redx Pharma PLC, Alderley Edge (GB)

(72) Inventors: Ian Cooper, Alderley Edge (GB); Amanda Lyons, Alderley Edge (GB)

(21) Appl. No.: 16/074,899

(22) PCT Filed: Feb. 8, 2017
(86) PCT No.: PCT/GB2017/050315

§ 371 Date: Aug. 2, 2018

(30) Foreign Application Priority Data
Feb. 8, 2016 (GB) 1602235.2

(51) Int. Cl. C07D 519/00; A61P 31/04

(52) U.S. Cl. CPC C07D 519/00; A61P 31/04

(57) ABSTRACT

This invention relates to antibacterial drug compounds containing a bicyclic core, typically a bicycle in which one of the rings is an oxazolidinone. It also relates to pharmaceutical formulations of antibacterial drug compounds. It also relates to uses of the derivatives in treating bacterial infections and in methods of treating bacterial infections.

This invention relates to antibacterial drug compounds containing a bicyclic core, typically a bicycle in which one of the rings is an oxazolidinone. It also relates to pharmaceutical formulations of antibacterial drug compounds. It also relates to uses of the derivatives in treating bacterial infections and in methods of treating bacterial infections. The invention is also directed to antibacterial drug compounds which are capable of treating bacterial infections which are currently hard to treat with existing drug compounds. Such infections are frequently referred to as resistant strains.

The increasing occurrence of bacterial resistance to antibiotics is viewed by many as being one of the most serious threats to human health. Multidrug resistance has become common among some pathogens, e.g. Staphylococcus aureus, Streptococcus pneumoniae, Clostridium difficile and Pseudomonas aeruginosa. Of these, Staphylococcus aureus, a Gram-positive bacterium, is the most concerning due to its potency and its capacity to adapt to environmental conditions. Methicillin resistant Staphylococcus aureus (MRSA) is probably the most well-known group of resistant strains and has reached pandemic proportions. Of particular concern is the increasing incidence of ‘community acquired’ infections, i.e. those occurring in subjects with no prior hospital exposure.

While less widespread, antibiotic resistant Gram-negative strains, such as either Escherichia coli NDM-1 (New Delhi metallo-β-lactamase 1) or Klebsiella pneumoniae NDM-1, are also very difficult to treat. Frequently only expensive antibiotics such as vancomycin and colistin are effective against these strains.

The fluoroquinolone antibacterial family are synthetic broad-spectrum antibiotics. They were originally introduced to treat Gram-negative bacterial infections, but are also used for the treatment of Gram-positive strains. One problem with existing fluoroquinolones can be the negative side effects that may sometimes occur as a result of their use. In general, the common side-effects are mild to moderate but, on occasion, more serious adverse effects occur. Some of the serious side effects that occur, and which occur more commonly with fluoroquinolones than with other antibiotic drug classes, include central nervous system (CNS) toxicity and cardiotoxicity. In cases of acute overdose there may be renal failure and seizure. In addition, an increasing number of strains of MRSA are also resistant to fluoroquinolone antibiotics, in addition to β-lactam antibiotics such as methicillin.

Gonorrhoea is a human sexually-transmitted infection (STI) caused by the Gram-negative bacterium Neisseria gonorrhoeae, a species of the genus Neisseria that also includes the pathogen N. meningitidis, which is one of the aetiological agents of meningitis. Untreated infection can result in a range of clinical complications including urethritis, dysuria, epididymitis, pelvic inflammatory disease, cervicitis, endometritis and even infertility and ectopic pregnancy. In rare cases, gonorrhoea can also spread to the blood to cause disseminated gonococcal infection that can manifest as arthritis, endocarditis or meningitis. Human immunodeficiency virus (HIV) is more readily-transmitted in individuals co-infected with gonorrhoea. Throughout the twentieth and twenty-first centuries gonorrhoea has been treated with a range of antibiotics. The sulfonamides were the first antibiotics used for the treatment of gonorrhoea, followed by penicillin, tetracycline and spectinomycin. In each case the development of resistance to these drugs by N. gonorrhoeae led to their use being discontinued. The fluoroquinolone antibiotics ciprofloxacin and ofloxacin were also historically recommended for the treatment of gonorrhoea. However, by 2007, fluoroquinolone resistance rates had reached 15% of gonococcal isolates and their use was abandoned. Current treatment recommendations comprise the cephalosporin antibiotics cefixime or ceftriaxone in combination with azithromycin or doxycycline. Resistance to cefixime and ceftriaxone has emerged in recent years. The CDC estimates that approximately 246,000 of the 820,000 gonococcal infections per year in the United States are drug-resistant (Antibiotic Resistance Threats in the United States, 2013, Centers for Disease Control and Prevention).

Another disease in which the development of resistance and multidrug resistance is of particular concern is tuberculosis (TB). From the 17^{th} century to the early-20^{th} century TB was one of the most common causes of death. The development of effective treatments and vaccinations during the mid-20^{th} century led to a sharp reduction in the number of deaths arising from the disease. TB is usually caused by Mycobacterium tuberculosis. Mycobacteria are aerobic bacteria and, as a result, tuberculosis infections most often develop in the lungs (pulmonary tuberculosis), although this is not always the case. Mycobacteria lack an outer cell membrane and as such they are often classified as Gram-positive bacteria, although they are in many ways atypical. They have a unique cell wall which provides protection against harsh conditions (e.g. acidic, oxidative) but also provides natural protection against many antibiotics. Other antibiotics, such as beta-lactams, are inactive against TB due to the intrinsic lack of activity of the compounds in the mycobacteria. Thus, a drug molecule may have excellent activity against other bacterial strains but no activity against wild-type TB. A number of TB-specific antibiotics have been developed, such as isoniazid, rifampicin, pyrazinamide and ethambutol and these are typically used in combination. Unfortunately, there is now increasing incidence of multidrug-resistant TB (MDR-TB). MDR-TB is the term typically used to refer to TB that has developed a resistance to isoniazid and rifampicin. MDR-TB can also be resistant to fluoroquinolones and also to the so-called ‘second-line’ injectable anti-TB drugs: kanamycin, capreomycin and amikacin. Where a strain of TB is resistant to isoniazid and rifampicin as well as one fluoroquinolone and one of the injectable anti-TB drugs, it is known as extensively drug resistant (XDR-TB). MDR-TB and XDR-TB are often found in those who have been previously treated for TB, but these forms of TB are just as infectious as wild-type TB and the incidence of MDR-TB and XDR-TB around the world is increasing. According to a 2013 World Health Organisation report, infections arising from XDR-TB had at that time been identified in 84 different countries. There have even been some reports of strains of TB which were resistant to all drugs tested against them (so-called ‘totally drug resistant tuberculosis’, TDR-TB). The ‘second-line’ anti-TB drugs and other antibiotics typically used to treat resistant infections can have unfavourable side effects.

Bacterial resistance is also becoming a problem in the treatment of animals. Antibacterials find widespread use in industrial farming, e.g. to prevent mastitis in dairy cattle, where they are often used prophylactically. Such widespread prophylactic use has led to the build-up of resistance in certain bacterial strains that are particularly relevant to animal health.

In spite of the numerous different antibiotics known in the art for a variety of different infections, there continues to be a need for antibiotics that can provide an effective treatment in a reliable manner. In addition, there remains a need for antibiotic drugs that can avoid or reduce the side-effects associated with known antibiotics.

It is an aim of certain embodiments of this invention to provide new antibiotics. In particular, it is an aim of certain embodiments of this invention to provide antibiotics that are active against resistant strains of Gram-positive and/or Gram-negative bacteria. It is an aim of certain embodiments of this invention to provide compounds that have activity that is comparable to those of existing antibiotics, and ideally which is better. It is an aim of certain embodiments of this invention to provide such activity against wild-type strains at the same time as providing activity against one or more resistant strains.

It is an aim of certain embodiments of this invention to provide compounds that exhibit a smaller decrease in activity against resistant strains compared to wild-type strains than prior art compounds do. It may be that certain compounds of the invention are less active than prior art compounds but there is a benefit associated with having a more consistent activity against a range of strains.

It is an aim of certain embodiments of this invention to provide antibiotics that exhibit reduced cytotoxicity relative to prior art compounds and existing therapies.

It is an aim of certain embodiments of this invention to provide treatment of bacterial infections that is effective in a selective manner at a chosen site of interest. Another aim of certain embodiments of this invention is to provide antibiotics having a convenient pharmacokinetic profile and a suitable duration of action following dosing. A further aim of certain embodiments of this invention is to provide antibiotics in which the metabolised fragment or fragments of the drug after absorption are GRAS (Generally Regarded As Safe).

Certain embodiments of the present invention satisfy some or all of the above aims.

### Compounds of the Invention

In a first aspect, the invention provides a compound of formula (I), or a pharmaceutically acceptable salt or N-oxide thereof:

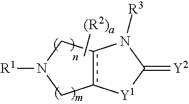

wherein [symbol] is a double bond or a single bond;

Y¹ is independently selected from O and S;

Y² is independently selected from O and S;

R¹ is independently selected from -L¹-Ar¹-Ar² and

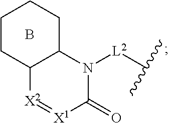

Ar¹ and Ar² are each independently selected from a phenyl or monocyclic heteroaryl group;

-L¹- is —C₁-C₃-alkylene-;

X¹ is independently selected from N and CR⁴ and X² is independently selected from N and CR⁵; or

X¹ and X² together form a 5-membered heteroaryl ring;

-L²- is —C₂-C₃-alkylene-;

Ring B is independently selected from: phenyl, monocyclic 6-membered heteroaryl and pyridinone, optionally substituted with a single —Y³—R⁶ group; Y³ is absent or is independently selected from NR⁷, O and S; where Ring B is a pyridinone ring, the nitrogen of the Ring B pyridinone may be attached to the proximal end of a —C₁-C₃-alkylene-group that is attached at its distal end to the group -L²-

R² is independently at each occurrence selected from: halo, nitro, cyano, NR⁸R⁹, NR⁸S(O)₂R⁸, NR⁸CONR⁸R⁸, NR⁸C(O)R⁸, NR⁸CO₂R⁸, OR⁸, SR⁸, SOR⁸, SO₃R⁸, SO₂R⁸, SO₂NR⁸R⁸, CO₂R⁸, C(O)R⁸, CONR⁸R⁸, C₁-C₄-alkyl, C₂-C₄-alkenyl, C₂-C₄-alkynyl, C₁-C₄-haloalkyl and O—C₁-C₄-haloalkyl;

R³ is a bicyclic carbocyclic or heterocyclic ring system in which at least one of the two rings is aryl or heteroaryl;

or R³ is -L³-phenyl; wherein -L³- is selected from —CR⁸═CR⁸— and —C₄-cycloalkyl-;

R⁴ and R⁵ are each independently selected from H, halo, cyano, C₁-C₄-alkyl and O—C₁-C₄-alkyl;

R⁶ is independently selected from: H, C₁-C₄-alkyl, C₂-C₄-alkenyl, C₂-C₄-alkynyl, C₁-C₄-haloalkyl, C₃-C₈-cycloalkyl, ₄₋₇-heterocycloalkyl, phenyl, monocyclic heteroaryl and C₁-C₃-alkylene-R^{6a}; wherein R^{6a} is independently selected from C₃-C₈-cycloalkyl, ₄₋₇-heterocycloalkyl, phenyl and monocyclic heteroaryl;

R⁷ is independently selected from: H and C₁-C₄-alkyl;

or R⁶ and R⁷ together with the nitrogen to which they are attached form a 4- to 7-membered heterocycloalkyl ring;

R⁸ is independently at each occurrence selected from: H and C₁-C₄-alkyl;

R⁹ is independently selected from: H, C₁-C₄-alkyl, C₁-C₄-haloalkyl, S(O)₂—C₁-C₄-alkyl and C(O)—C₁-C₄-alkyl;

a is an integer from 0 to 4;

n and m are each an integer selected from 1 and 2; wherein the sum of n and m is 2 or 3;

wherein any of the aforementioned alkyl, alkylene, alkenyl, alkynyl, haloalkyl, cycloalkyl, carbocyclic, heterocyclic, heterocycloalkyl, aryl, phenyl and heteroaryl groups is optionally substituted, where chemically possible, by 1 to 5 substituents which are each independently at each occurrence selected from the group consisting of: oxo, ═NR^{a}, ═NOR^{a}, halo, nitro, cyano, NR^{a}R^{a}, NR^{a}S(O)₂R^{a}, NR^{a}C(O)R^{a}, NR^{a}CONR^{a}R^{a}, NR^{a}CO₂R^{a}, OR^{a}; SR^{a}, SOR^{a}, SO₃R^{a}, SO₂R^{a}, SO₂NR^{a}R^{a}, CO₂R^{a}, C(O)R^{a}, CONR^{a}R^{a}, CR^{a}R^{a}NR^{a}R^{a}, CR^{a}R^{a}OR^{a}, C₁-C₄-alkyl, C₂-C₄-alkenyl, C₂-C₄-alkynyl, C₁-C₄-haloalkyl and O—C₁-C₄-haloalkyl; wherein R^{a} is independently at each occurrence selected from H, C₁-C₄ alkyl.

In an embodiment, the compound of formula (I) is a compound of formula (II):

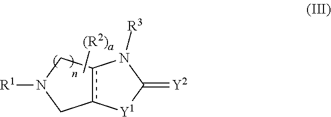

wherein [symbol] R¹, R², R³, Y¹, Y², n and a are as defined above for formula (I).

In an embodiment, the compound of formula (I) is a compound of formula (III):

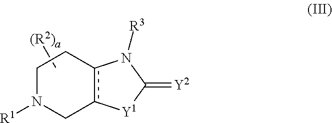

wherein [symbol] R¹, R², R³, Y¹, Y² and a are as defined above for formula (I).

In an embodiment, the compound of formula (I) is a compound of formula (IV):

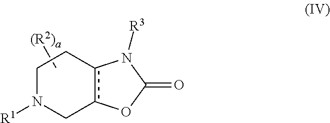

wherein [symbol] R¹, R², R³ and a are as defined above for formula (I).

In an embodiment, the compound of formula (I) is a compound of formula (V):

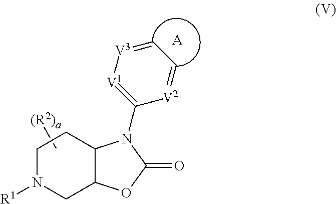

wherein R¹, R² and a are as defined above for formula (I); V¹, V² and V³ are each independently selected from: N and CR¹⁰; with the proviso that no more than two of V¹, V² and V³ are N; and wherein the ring A is a substituted or unsubstituted 5- or 6-membered saturated cycloalkyl or heterocycloalkyl ring; and R¹⁰ is independently at each occurrence selected from H, halo, nitro, cyano, NR^{a}R^{a}, NR^{a}S(O)₂R^{a}, NR^{a}C(O)R^{a}, NR^{a}CONR^{a}R^{a}, NR^{a}CO₂R^{a}, OR^{a}; SR^{a}, SOR^{a}, SO₃R^{a}, SO₂R^{a}, SO₂NR^{a}R^{a}, CO₂R^{a}, C(O)R^{a}, CONR^{a}R^{a}, CR^{a}R^{a}NR^{a}R^{a}, CR^{a}R^{a}OR^{a}, C₁-C₄-alkyl, C₂-C₄-alkenyl, C₂-C₄-alkynyl and C₁-C₄-haloalkyl.

In an embodiment, the compound of formula (I) is a compound of formula (VI):

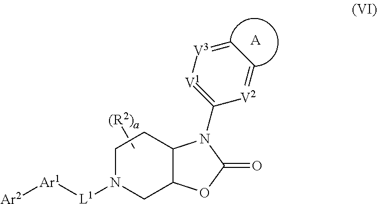

wherein Ar¹, Ar², R², L¹ and a are as defined above for formula (I) and wherein V¹, V² and V³ and ring A are as described above for formula (V).

In an embodiment, the compound of formula (I) is a compound of formula (VII):

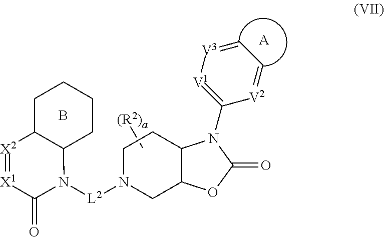

wherein R², L², X¹, X², Ring B and a are as defined above for formula (I) and wherein V¹, V² and V³ and ring A are as described above for formula (V).

In an embodiment, the compound of formula (I) is a compound of formula (VIII):

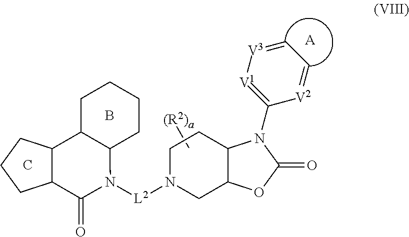

wherein R², L², Ring B and a are as defined above for formula (I) and wherein V¹, V² and V³ and ring A are as described above for formula (V) and wherein ring C is a 5-membered heteroaryl ring.

In an embodiment, the compound of formula (I) is a compound of formula (IX):

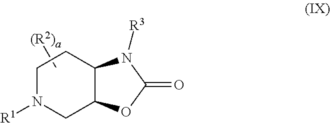

wherein R¹, R², R³ and a are as defined above for formula (I). For the absence of doubt the hashed and solid wedges in formula (IX) are intended to depict the relative stereochemistry of the indicated bonds and not the absolute stereochemistry, i.e. the compound may be in the form of a single enantiomer or in the form of a racemate.

In an embodiment, the compound of formula (I) is a compound of formula (IX):

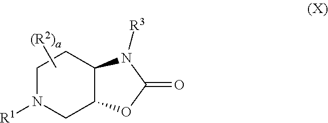

wherein R¹, R², R³ and a are as defined above for formula (I). For the absence of doubt the hashed and solid wedges in formula (X) are intended to depict the relative stereochemistry of the indicated bonds and not the absolute stereochemistry, i.e. the compound may be in the form of a single enantiomer or in the form of a racemate.

The following statements apply to compounds of any of formulae (I) to (X). These statements are independent and interchangeable. In other words, any of the features described in any one of the following statements may (where chemically allowable) be combined with the features described in one or more other statements below. In particular, where a compound is exemplified or illustrated in this specification, any two or more of the statements below which describe a feature of that compound, expressed at any level of generality, may be combined so as to represent subject matter which is contemplated as forming part of the disclosure of this invention in this specification.

[symbol] may be a double bond. Typically, however, [symbol] is a single bond.

Where [symbol] is a single bond, it may be that the groups NR³ and Y¹ are orientated cis to each other. Alternatively, it may be that the groups NR³ and Y¹ are orientated trans to each other.

Y¹ may be S. Preferably, however, Y¹ is O.

Y² may be S. Preferably, however, Y² is O.

m may be 1. m may be 2. n may be 1. n may be 2. Preferably, the sum of m and n is 3. Thus, it may be that m is 1 and n is 2.

R¹ may be:

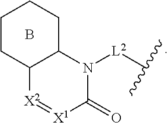

Throughout this specification, the group

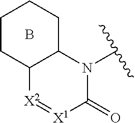

may be referred to as R^{1a}. Thus, R¹ may be -L²-R^{1a}.

-L². may be —C₂-C₃-alkylene-. -L². may be —C₂-alkylene-. -L². may be —C₃-alkylene-. -L²- may be substituted with 1 or 2 groups selected from ═O, methyl, CH₂OH, CO₂R^{a}, and CO₂NR^{a}R^{a}. It may be, however, that -L²- is unsubstituted alkylene. Thus, -L¹- may be —CH₂CH₂—.

X¹ may be N. Thus, R^{1a} may have the structure:

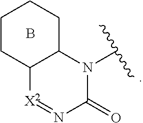

Alternatively, X¹ may be CR⁴. Thus, R^{1a} may have the structure:

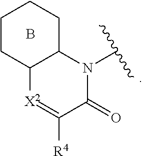

X² may be N. Thus, R^{1a} may have the structure:

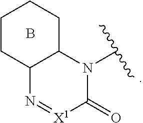

Alternatively, X² may be CR⁵. Thus, R^{1a} may have the structure:

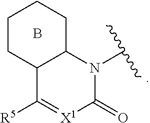

It may be that X¹ is N and X² is CR⁵. Thus, R^{1a} may have the structure:

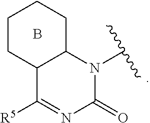

It may be that X¹ is N and X² is N. Thus, R^{1a} may have the structure:

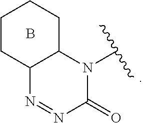

It may be that X¹ is CR⁴ and X² is N. Thus, R^{1a} may have the structure:

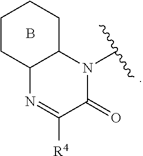

It may be that X¹ is CR⁵ and X² is CR⁶. Thus, R^{1a} may have the structure:

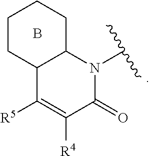

It may be that R⁴ and R⁵ are each independently selected from H, halo, cyano, C₁-C₄-alkyl and O—C₁-C₄-alkyl; or R⁴ and R⁵ together with the carbons to which they are attached together form a 5-membered heteroaryl ring. Compounds in which R⁴ and R⁵ together with the carbons to which they are attached together form a 5-membered heteroaryl ring are examples of compounds is which X¹ and X² together form a 5-membered heteroaryl group.

It may be that R⁴ is independently selected from H or C₁-C₄-alkyl. It may be that R⁴ is H. It may be that R⁵ is independently selected from H or C₁-C₄-alkyl. It may be that R⁵ is H.

It may be that R⁴ and R⁵ are each independently selected from: H, halo, cyano, C₁-C₄-alkyl and O—C₁-C₄-alkyl. It may be that R⁴ and R⁵ are each independently selected from H, C₁-C₄-alkyl and O—C₁-C₄-alkyl or that R⁴ and R⁵, together with the carbons to which they are attached together form a 5-membered heteroaryl ring. It may be that R⁴ and R⁵ are each independently at each occurrence selected from H or C₁-C₄-alkyl. It may be that R⁴ and R⁵ are at each occurrence H.

Alternatively, it may be that R⁴ and R⁵, together with the carbons to which they are attached together form a 5-membered heteroaryl ring. Exemplary heteroaryl rings include oxazole, thiazole, isoxazole, isothiazole, pyrazole, imidazole, triazole, pyrole, thiophene, furan and oxadiazole. For the absence of doubt, the double bond depicted in the structure above between X¹ and X² may be delocalised into the heteroaromatic ring.

Thus, R^{1a} may have the structure:

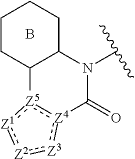

wherein Z⁴ and Z⁵ are each independently selected from C and N; Z¹, Z² and Z³ are each independently selected from O, S, S(O), NR^{a} and CR¹¹; wherein the ring formed by Z¹, Z², Z³, Z⁴ and Z⁵ contains two endocyclic double bonds and with the further proviso that at least one of Z¹, Z², Z³, Z⁴ and Z⁵ is O, S, N or NR^{a}; and wherein R¹¹ is independently selected from H, C₁-C₄-alkyl, CR^{a}R^{a}OR^{a}, CR^{a}R^{a}NR^{a}R^{a}, CO₂R^{a} and CONR^{a}R^{a}.

In certain examples, the heteroaryl ring may be a ring selected from oxazole, thiazole, isoxazole and isothiazole. Thus, R^{1a} may have the structure:

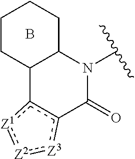

wherein one of Z¹, Z² and Z³ is N, one of Z¹, Z² and Z³ is CR¹¹ and the final one of Z¹, Z² and Z³ is selected from O and S; provided that the ring comprising Z¹, Z² and Z³ contains two endocyclic double bonds; and wherein R¹¹ is independently selected from H, C₁-C₄-alkyl, CR^{a}R^{a}OR^{a}, CR^{a}R^{a}NR^{a}R^{a}, CO₂R^{a} and CONR^{a}R^{a}.

Thus, R^{1a} may have the structure:

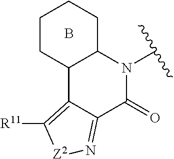

wherein Z² is independently selected from O and S. Z² may be O. Z² may be S.

Ring B may be selected from a phenyl ring or a 6-membered heteroaryl ring. Thus, Ring B may be a phenyl ring. Ring B may be a pyridyl ring.

R^{1a} may have the structure:

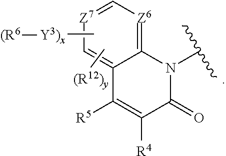

wherein x is 0 or 1; y is an integer from 0 to 2; Z⁶ and Z⁷ are each independently selected from carbon or nitrogen; and R¹² is independently selected from halo, nitro, cyano, NR^{a}R^{a}, NR^{a}S(O)₂R^{a}, NR^{a}C(O)R^{a}, NR^{a}CONR^{a}R^{a}, NR^{a}CO₂R^{a}, OR^{a}; SR^{a}, SOR^{a}, SO₃R^{a}, SO₂R^{a}, SO₂NR^{a}R^{a}, CO₂R^{a}, C(O)R^{a}, CONR^{a}R^{a}, CR^{a}R^{a}NR^{a}R^{a}, CR^{a}R^{a}OR^{a}, C₁-C₄-alkyl, C₂-C₄-alkenyl, C₂-C₄-alkynyl and C₁-C₄-haloalkyl.

Thus, R^{1a} may have the structure:

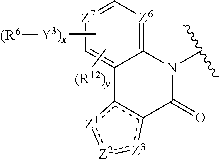

wherein one of Z¹, Z² and Z³ is N, one of Z¹, Z² and Z³ is CR¹¹ and the final one of Z¹, Z² and Z³ is selected from O and S; provided that the ring comprising Z¹, Z² and Z³ contains two endocyclic double bonds.

Likewise, R^{1a} may have the structure:

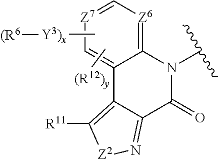

wherein Z² is independently selected from O and S. Z² may be O. Z² may be S.

x may be 0. Thus it may be that there is no Y³—R⁶ group on R¹.

Alternatively, x may be 1.

Thus, R^{1a} may have the structure:

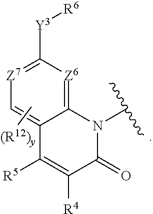

R^{1a} may have the structure:

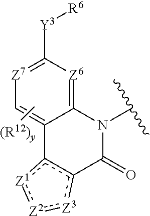

wherein one of Z¹, Z² and Z³ is N, one of Z¹, Z² and Z³ is CR¹¹ and the final one of Z¹, Z² and Z³ is selected from O and S; provided that the ring comprising Z¹, Z² and Z³ contains two endocyclic double bonds.

Likewise, R^{1a} may have the structure:

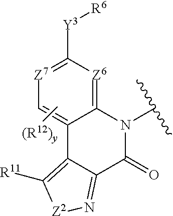

wherein Z² is independently selected from O and S. Z² may be O. Z² may be S.

Likewise, R^{1a} may have the structure:

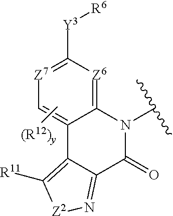

wherein Z² is independently selected from O and S. Z² may be O. Z² may be S.

R^{1a} may have the structure:

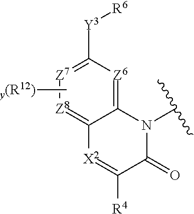

wherein y is an integer from 0 to 2; Z⁶, Z⁷ and Z⁸ are each independently selected from carbon or nitrogen; providing that no more than 2 of Z⁶, Z⁷ and Z are nitrogen; and R¹² is independently selected from halo, nitro, cyano, NR^{a}R^{a}, NR^{a}S(O)₂R^{a}, NR^{a}C(O)R^{a}, NR^{a}CONR^{a}R^{a}, NR^{a}CO₂R^{a}, OR^{a}; SR^{a}, SOR^{a}, SO₃R^{a}, SO₂R^{a}, SO₂NR^{a}R^{a}, CO₂R^{a}, C(O)R^{a}, CONR^{a}R^{a}, CR^{a}R^{a}NR^{a}R^{a}, CR^{a}R^{a}OR^{a}, C₁-C₄-alkyl, C₂-C₄-alkenyl, C₂-C₄-alkynyl, C₁-C₄-haloalkyl and O—C₁-C₄-haloalkyl.

y may be 0.

Z⁶ may be nitrogen. Alternatively, Z⁶ may be carbon. Z⁷ may be nitrogen. Alternatively, Z⁷ may be carbon. It may be that Z⁶ and Z⁷ are each carbon. It may be that Z⁶ and Z⁷ are each nitrogen. It may be that Z⁶ is nitrogen and Z⁷ is carbon

R¹¹ may be selected from H and C₁-C₄-alkyl. In certain particular embodiments, R¹¹ is H.

If present, R¹² may at each occurrence be selected from halo and C₁-C₄-alkyl.

Y³ is preferably O.

It may be that R⁶ and R⁷ together with the nitrogen to which they are attached form a 4- to 7-membered heterocycloalkyl ring. It may be that R⁶ and R⁷ together with the nitrogen to which they are attached form a 6-membered heterocycloalkyl ring, e.g. a piperidine, morpholine or piperazine ring.

Preferably, however, R⁶ is independently selected from: H, C₁-C₄-alkyl, C₃-C₈-cycloalkyl, ₄₋₇-heterocycloalkyl, phenyl and monocyclic heteroaryl. R⁶ may be independently selected from: C₁-C₄-alkyl, phenyl and monocyclic heteroaryl.

R⁶ may be alkyl. R⁶ may be selected from C₁-C₄-alkyl. Alternatively, R⁶ may be selected from phenyl and monocyclic heteroaryl. R⁶ may be phenyl. R⁶ may be unsubstituted phenyl or R⁶ may be substituted phenyl. R⁶ may be monocyclic heteroaryl, e.g. a 6-membered heteroaryl group. Thus, R⁶ may be pyridyl, e.g. unsubstituted pyridyl. R⁶ may be 3-pyridyl, e.g. unsubstituted 3-pyridyl. R⁶ may be C₃-C₈-cycloalkyl, e.g. cyclohexyl. R⁶ may be ₄₋₇-heterocycloalkyl, e.g. piperidine or tetrahydropyran.

It may be that Y³ is O and R⁶ is independently selected from: C₁-C₄-alkyl, C₂-C₄-alkenyl, C₂-C₄-alkynyl, C₁-C₄-haloalkyl, C₃-C₈-cycloalkyl, ₄₋₇-heterocycloalkyl, phenyl, monocyclic heteroaryl and C₁-C₃-alkylene-R^{6a}; wherein R^{6a} is independently selected from C₃-C₈-cycloalkyl, ₄₋₇-heterocycloalkyl, phenyl and monocyclic heteroaryl.

Exemplary R¹ groups include:

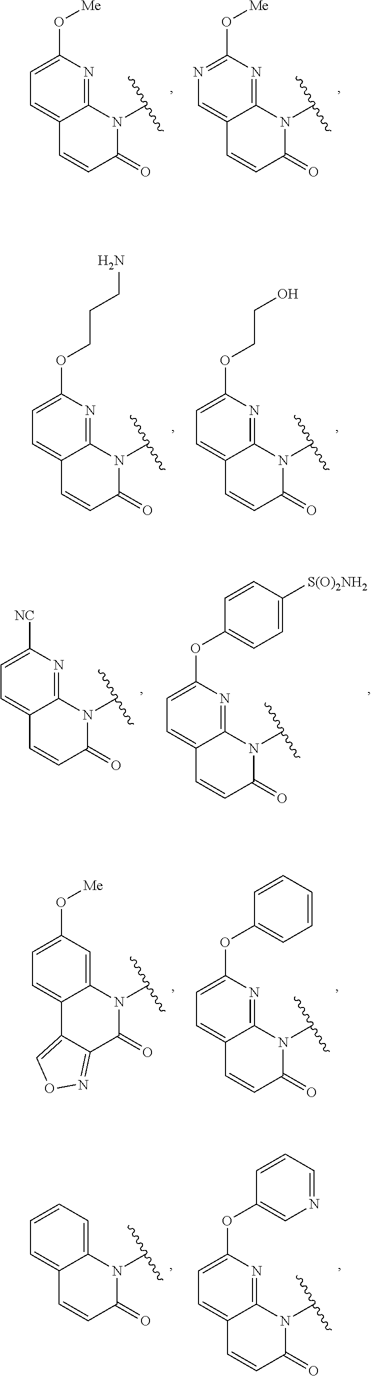

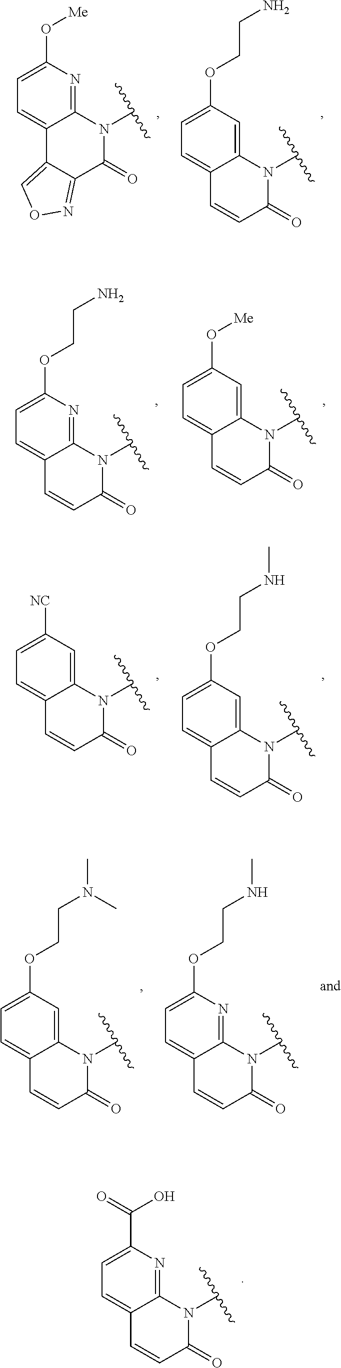

In these embodiments, -L²- is typically an ethylene group.

It may be that R¹ is -L¹-Ar¹-Ar²; wherein Ar¹ is independently selected from a phenyl or monocyclic heteroaryl group; and wherein Ar² is a monocyclic heteroaryl group.

-L¹- may be —C₁-C₂-alkylene-. -L¹- may be —C₁-alkylene-. -L¹- may be —C₂-alkylene-. -L-may be substituted with 1 or 2 groups selected from ═O, methyl, CH₂OH, CO₂R^{a}, and CO₂NR^{a}R^{a}. It may be, however, that -L¹- is unsubstituted alkylene. Thus, -L¹- may be —CH₂—.

It may be that -L¹- and R⁴ together with the nitrogen to which they are attached form a 4- to 7-membered heterocyclic ring. Thus, it may be that -L¹- and R⁴ together with the nitrogen to which they are attached form a 4- to 5-membered heterocyclic ring. -L¹- and R⁴ together with the nitrogen to which they are attached may form a pyrrolidine or azetidine ring.

It may be that at least one of Ar¹ and Ar² is a monocyclic heteroaryl group, e.g. a 6-membered monocyclic heteroaryl group, e.g. a pyridine. It may be that a single one of Ar¹ and Ar² is a monocyclic heteroaryl group, e.g. a 6-membered monocyclic heteroaryl group, e.g. a pyridine. It may be that a single one of Ar¹ and Ar² is phenyl, e.g. substituted phenyl.

It may be that Ar¹ is a phenyl group, e.g. a substituted phenyl group and Ar² is a 6-membered heteroaryl group, e.g. pyridine. It may be that Ar¹ is a 6-membered heteroaryl group, e.g. pyridine, and Ar² is a phenyl group.

Ar¹ may be a monocyclic heteroaryl group, e.g. a 6-membered monocyclic heteroaryl group. Ar¹ may be pyridine. Ar¹ may be a phenyl group. Ar¹ may be unsubstituted. Ar¹ may be substituted, e.g. Ar¹ may be substituted with a single hydroxyl group.

Thus, Ar¹ may have the structure:

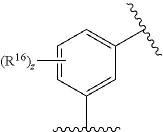

wherein z is an integer from 0 to 4; and R¹⁶ is independently selected from halo, nitro, cyano, NR^{a}R^{a}, NR^{a}S(O)₂R^{a}, NR^{a}C(O)R^{a}, NR^{a}CONR^{a}R^{a}, NR^{a}CO₂R^{a}, OR^{a}; SR^{a}, SOR^{a}, SO₃R^{a}, SO₂R^{a}, SO₂NR^{a}R^{a}, CO₂R^{a}, C(O)R^{a}, CONR^{a}R^{a}, CR^{a}R^{a}NR^{a}R^{a}, CR^{a}R^{a}OR^{a}, C₁-C₄-alkyl, C₂-C₄-alkenyl, C₂-C₄-alkynyl and C₁-C₄ haloalkyl. Ar¹ may have the structure:

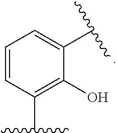 [SMILES: *c1cccc(*)c1O]

Ar² may be a monocyclic heteroaryl group. Ar² may be 6-membered heteroaryl group, e.g. a pyridyl group.

Ar² may have the structure:

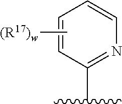

wherein w is an integer from 0 to 4; and R¹⁷ is independently selected from halo, nitro, cyano, NR^{a}R^{a}, NR^{a}S(O)₂R^{a}, NR^{a}C(O)R^{a}, NR^{a}CONR^{a}R^{a}, NR^{a}CO₂R^{a}, OR^{a}; SR^{a}, SOR^{a}, SO₃R^{a}, SO₂R^{a}, SO₂NR^{a}R^{a}, CO₂R^{a}, C(O)R^{a}, CONR^{a}R^{a}, CR^{a}R^{a}NR^{a}R^{a}, CR^{a}R^{a}OR^{a}, C₁-C₄-alkyl, C₂-C₄-alkenyl, C₂-C₄-alkynyl and C₁-C₄-haloalkyl. Ar¹ may have the structure:

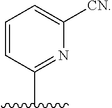 [SMILES: *c1cccc(C#N)n1]

Exemplary examples of Ar¹-Ar² include:

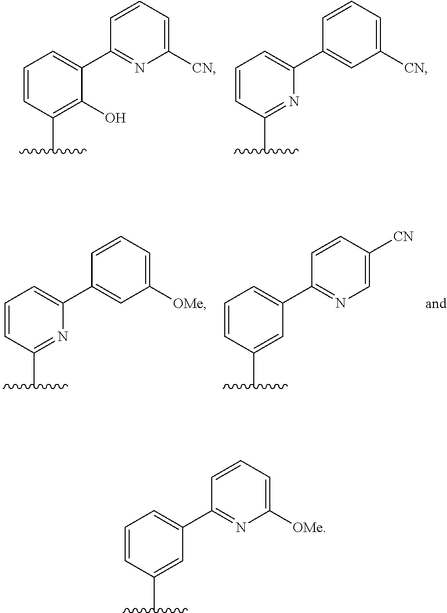

In these embodiments, -L¹- is typically a methylene group.

R² may be independently at each occurrence selected from: CO₂R⁸, C(O)R⁸, CONR⁸R⁸, C₁-C₄-alkyl, C₂-C₄-alkenyl, C₂-C₄-alkynyl and C₁-C₄-haloalkyl. a may be 0.

It may be that R³ is a bicyclic carbocyclic or heterocyclic ring system in which at least one of the two rings is aryl or heteroaryl.

Thus, R³ may take the form:

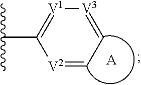

wherein V¹, V² and V³ are each independently selected from: N and CR¹⁰; with the proviso that no more than two of V¹, V² and V³ are N; and wherein the ring A is a substituted or unsubstituted 5- or 6-membered saturated cycloalkyl or heterocycloalkyl ring; and wherein R¹⁰ is independently at each occurrence selected from H, halo, nitro, cyano, NR^{a}R^{a}, NR^{a}S(O)₂R^{a}, NR^{a}C(O)R^{a}, NR^{a}CONR^{a}R^{a}, NR^{a}CO₂R^{a}, OR^{a}; SR^{a}, SOR^{a}, SO₃R^{a}, SO₂R^{a}, SO₂NR^{a}R^{a}, CO₂R^{a}, C(O)R^{a}, CONR^{a}R^{a}, CR^{a}R^{a}NR^{a}R^{a}, CR^{a}R^{a}OR^{a}, C₁-C₄-alkyl, C₂-C₄-alkenyl, C₂-C₄-alkynyl and C₁-C₄-haloalkyl.

Preferably, R³ takes the form:

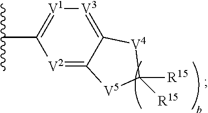

wherein V⁴ and V⁵ are each independently selected from O, S and NR^{a}; R¹⁵ is independently at each occurrence selected from: H, fluoro, cyano, CO₂R^{a}, C(O)R^{a}, CONR^{a}R^{a}, C₁-C₄-alkyl, C₂-C₄-alkenyl, C₂-C₄-alkynyl and C₁-C₄-haloalkyl; or any two R¹⁵ groups which are attached to the same carbon together form a group selected from: ═O, ═NR^{a} and ═NOR^{a}; and b is an integer selected from 1 and 2. For the absence of doubt, V¹, V², V³, V⁴, V⁵, R¹⁵ and b are selected such that the number of substituent groups (as defined above in relation to formula (I)) off the R³ bicycle does not exceed 5.

It may be that V¹, V² and V³ are each independently selected from: N and CH; with the proviso that no more than two of V¹, V² and V³ are N. It may be that a single one of V¹, V² and V³ is N. Preferably, V³ is CR¹⁰ (e.g. CH). Thus, it may be that V¹ is N and V² is CR¹⁰ (e.g. CH). Alternatively, it may be that V² is N and V¹ is CR¹⁰ (e.g. CH). In a further alternative, it may be that V¹ and V² are each N.

R¹⁵ may be independently at each occurrence selected from: H, fluoro, cyano, CO₂R^{a}, C(O)R^{a}, CONR^{a}R^{a}, C₁-C₄-alkyl, C₂-C₄-alkenyl, C₂-C₄-alkynyl and C₁-C₄-haloalkyl; or any two R¹⁵ groups which are attached to the same carbon together form a group selected from: ═O, ═NR^{a} and ═NOR^{a}. Preferably R¹⁵ is independently at each occurrence selected from H, F, C₁-C₄-alkyl or C₁-C₄-haloalkyl; or any two R¹⁵ groups which are attached to the same carbon together form a ═O group. Preferably R¹⁵ is independently at each occurrence selected from: H, C₁-C₄-alkyl or C₁-C₄-haloalkyl; or any two R¹⁵ groups which are attached to the same carbon together form a ═O group.

In a preferred embodiment, V⁴ is O. Thus, it may be that both V⁴ and V⁵ are 0. It may be that V⁴ is O and V⁵ is S. It may be that V⁴ is O and V⁵ is NR^{a} (e.g. NH).

V⁴ can also be S. Thus, it may be that V⁴ is S and V⁵ is NR^{a} (e.g. NH).

It may be that V⁵ is NR^{a} (e.g. NH). In this case it is preferable that the —CR¹⁵R¹⁵— group attached to said V⁵ is C═O.

b may be 1. Preferably, b is 2.

In a specific embodiment, V⁴ is O, V⁵ is O, b is 2 and R¹⁵ is at each occurrence H. In another specific embodiment, V⁴ is O, V⁵ is S, b is 2 and R¹⁵ is at each occurrence H. In yet another specific embodiment, V⁴ is O, V⁵ is NH, b is 2, the —CR¹⁵R¹⁵— group attached to V⁵ is C═O and the —CR¹⁵R¹⁵— group attached to V⁴ is CH₂.

R³ may take the form:

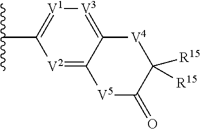

wherein V¹, V² and V³ are each independently selected from: N and CR¹⁰; with the proviso that no more than two of V¹, V² and V³ are N; V⁴ and V⁵ are each independently selected from O, S and NR^{a}; wherein R¹⁰ is independently at each occurrence selected from H, halo, nitro, cyano, NR^{a}R^{a}, NR^{a}S(O)₂R^{a}, NR^{a}C(O)R^{a}, NR^{a}CONR^{a}R^{a}, NR^{a}CO₂R^{a}, OR^{a}; SR^{a}, SOR^{a}, SO₃R^{a}, SO₂R^{a}, SO₂NR^{a}R^{a}, CO₂R^{a}, C(O)R^{a}, CONR^{a}R^{a}, CR^{a}R^{a}NR^{a}R^{a}, CR^{a}R^{a}OR^{a}, C₁-C₄-alkyl, C₂-C₄-alkenyl, C₂-C₄-alkynyl and C₁-C₄-haloalkyl; and R¹⁵ is independently at each occurrence selected from: H, fluoro, cyano, CO₂R^{a}, C(O)R^{a}, CONR^{a}R^{a}, C₁-C₄-alkyl, C₂-C₄-alkenyl, C₂-C₄-alkynyl and C₁-C₄-haloalkyl.

R³ may take the form:

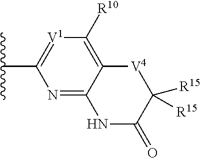

wherein V¹ is are each independently selected from: N and CR¹⁰; V⁴ is independently selected from O and S; wherein R¹⁰ is independently at each occurrence selected from H, halo, nitro, cyano, NR^{a}R^{a}, NR^{a}S(O)₂R^{a}, NR^{a}C(O)R^{a}, NR^{a}CONR^{a}R^{a}, NR^{a}CO₂R^{a}, OR^{a}; SR^{a}, SOR^{a}, SO₃R^{a}, SO₂R^{a}, SO₂NR^{a}R^{a}, CO₂R^{a}, C(O)R^{a}, CONR^{a}R^{a}, CR^{a}R^{a}NR^{a}R^{a}, CR^{a}R^{a}OR^{a}, C₁-C₄-alkyl, C₂-C₄-alkenyl, C₂-C₄-alkynyl and C₁-C₄-haloalkyl; and R¹⁵ is independently at each occurrence selected from: H, fluoro, cyano, CO₂R^{a}, C(O)R^{a}, CONR^{a}R^{a}, C₁-C₄-alkyl, C₂-C₄-alkenyl, C₂-C₄-alkynyl and C₁-C₄-haloalkyl.

Exemplary R³ groups include:

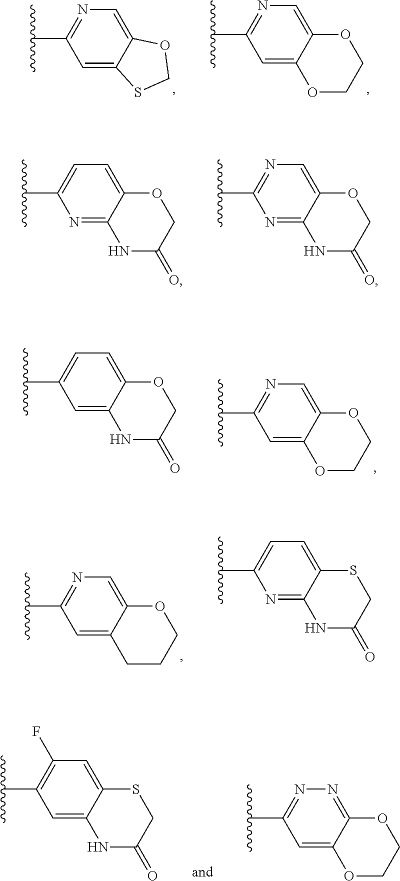

R³ may be -L³-phenyl.

R³ may take the form,

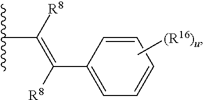

wherein R¹⁶ is independently at each occurrence selected from halo, nitro, cyano, NR^{a}R^{a}, NR^{a}S(O)₂R^{a}, NR^{a}C(O)R^{a}, NR^{a}CONR^{a}R^{a}, NR^{a}CO₂R^{a}, OR^{a}; SR^{a}, SOR^{a}, SO₃R^{a}, SO₂R^{a}, SO₂NR^{a}R^{a}, CO₂R^{a}, C(O)R^{a}, CONR^{a}R^{a}, CR^{a}R^{a}NR^{a}R^{a}, CR^{a}R^{a}OR^{a}, C₁-C₄-alkyl, C₂-C₄-alkenyl, C₂-C₄-alkynyl and C₁-C₄-haloalkyl; and u is an integer from 0 to 5.

R⁸ may at each occurrence be H.

R³ may take the form:

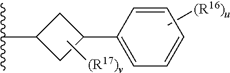

wherein R¹⁶ is independently at each occurrence selected from halo, nitro, cyano, NR^{a}R^{a}, NR^{a}S(O)₂R^{a}, NR^{a}C(O)R^{a}, NR^{a}CONR^{a}R^{a}, NR^{a}CO₂R^{a}, OR^{a}; SR^{a}, SOR^{a}, SO₃R^{a}, SO₂R^{a}, SO₂NR^{a}R^{a}, CO₂R^{a}, C(O)R^{a}, CONR^{a}R^{a}, CR^{a}R^{a}NR^{a}R^{a}, CR^{a}R^{a}OR^{a}, C₁-C₄-alkyl, C₂-C₄-alkenyl, C₂-C₄-alkynyl and C₁-C₄-haloalkyl; R¹⁷ is independently at each occurrence selected from oxo, fluoro, cyano, CO₂R^{a} C(O)R^{a}, CONR^{a}R^{a}, C₁-C₄-alkyl, C₂-C₄-alkenyl, C₂-C₄-alkynyl and C₁-C₄-haloalkyl; u is an integer from 0 to 5; and v is an integer from 0 to 4;

R¹⁷ may be selected from: fluoro, cyano, CO₂R^{a} C(O)R^{a}, CONR^{a}R^{a}, C₁-C₄-alkyl, C₂-C₄-alkenyl, C₂-C₄-alkynyl and C₁-C₄-haloalkyl.

v may be 0.

R¹⁶ may be independently at each occurrence selected from C₁-C₄-alkyl, halo, nitro and cyano.

u may be an integer from 1 to 5, e.g. from 1 to 3.

R³ may also take the form

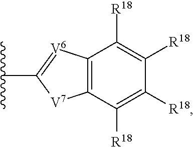

wherein V⁶ is independently selected from N and CR¹⁰ (e.g. CH); V⁷ is independently selected from NR^{a}, S and O; and R¹⁸ is independently at each occurrence selected from: H, halo, nitro, cyano, NR^{a}R^{a}, NR^{a}S(O)₂R^{a}, NR^{a}C(O)R^{a}, NR^{a}CONR^{a}R^{a}, NR^{a}CO₂R^{a}, NR^{a}C(O)R^{a}, OR^{a}; SR^{a,} SOR^{a}, SO₃R^{a}, SO₂R^{a}, SO₂NR^{a}R^{a}, CO₂R^{a} C(O)R^{a}, CONR^{a}R^{a}, C₁-C₄-alkyl, C₂-C₄-alkenyl, C₂-C₄-alkynyl, C₁-C₄-haloalkyl, and CR^{a}R^{a}NR^{a}R^{a}. R¹⁸ may be independently at each occurrence selected from H, F, CN, OR^{a}, nitro, C₁-C₄-alkyl, C₂-C₄-alkenyl, C₂-C₄-alkynyl and C₁-C₄-haloalkyl. For the absence of doubt, V⁶, V⁷ and R¹⁸ are selected such that the number of substituent groups (as defined above in relation to formula (I)) off the R³ bicycle does not exceed 5.

R³ may take the form

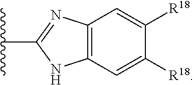

An exemplary R³ group is

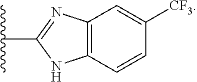 [SMILES: *c1nc2cc(C(F)(F)F)ccc2[nH]1]

The compound may be any one or more compound(s) selected from those made in Examples 1 to 10 and tested in Examples 11 and 12, or a pharmaceutically acceptable salt or N-oxide thereof.

The compound may be as described in one of the following numbered clauses:
1. A compound of formula (I), or a pharmaceutically acceptable salt or N-oxide thereof:

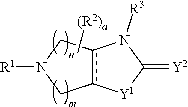

wherein [symbol] is a double bond or a single bond;

Y¹ is independently selected from O and S;

Y² is independently selected from O and S;

R¹ is independently selected from -L¹-Ar¹-Ar² and

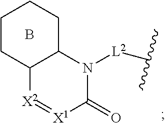

Ar¹ is independently selected from a phenyl or monocyclic heteroaryl group;

Ar² is a monocyclic heteroaryl group;

-L¹- is —C₁-C₃-alkylene-;

X¹ is independently selected from N and CR⁴ and X² is independently selected from N and CR⁵; or

X¹ and X² together form a 5-membered heteroaryl ring;

-L²- is —C₂-C₃-alkylene-;

Ring B is independently selected from: phenyl, monocyclic 6-membered heteroaryl and pyridinone, optionally substituted with a single —Y³—R⁶ group; Y³ is independently selected from NR⁷, O and S; where Ring B is a pyridinone ring, the nitrogen of the Ring B pyridinone may be attached to the proximal end of a —C₁-C₃-alkylene-group that is attached at its distal end to the group -L²-

R² is independently at each occurrence selected from: halo, nitro, cyano, NR⁸R⁹, NR⁸S(O)₂R⁸, NR⁸CONR⁸R⁸, NR⁸C(O)R⁸, NR⁸CO₂R⁸, OR⁸, SR⁸, SOR⁸, SO₃R⁸, SO₂R⁸, SO₂NR⁸R⁸, CO₂R⁸, C(O)R⁸, CONR⁸R⁸, C₁-C₄-alkyl, C₂-C₄-alkenyl, C₂-C₄-alkynyl and C₁-C₄-haloalkyl;

R³ is a bicyclic carbocyclic or heterocyclic ring system in which at least one of the two rings is aryl or heteroaryl;

or R³ is -L³-phenyl; wherein -L³- is selected from —CR⁸═CR⁸— and —C₄-cycloalkyl-; R⁴ and R⁵ are each independently selected from H, halo, cyano, C₁-C₄-alkyl and O—C₁-C₄-alkyl;

R⁶ is independently selected from: H, C₁-C₄-alkyl, C₃-C₈-cycloalkyl, ₄₋₇-heterocycloalkyl, phenyl and monocyclic heteroaryl;

R⁷ is independently selected from: H and C₁-C₄-alkyl;

or R⁶ and R⁷ together with the nitrogen to which they are attached form a 4- to 7-membered heterocycloalkyl ring;

R⁸ is independently at each occurrence selected from: H and C₁-C₄-alkyl;

R⁹ is independently selected from: H, C₁-C₄-alkyl, C₁-C₄-haloalkyl, S(O)₂—C₁-C₄-alkyl and C(O)—C₁-C₄-alkyl;

a is an integer from 0 to 4;

n and m are each an integer selected from 1 and 2; wherein the sum of n and m is 2 or 3; wherein any of the aforementioned alkyl, alkylene, alkenyl, alkynyl, haloalkyl, cycloalkyl, carbocyclic, heterocyclic, heterocycloalkyl, aryl, phenyl and heteroaryl groups is optionally substituted, where chemically possible, by 1 to 5 substituents which are each independently at each occurrence selected from the group consisting of: oxo, ═NR^{a}, ═NOR^{a}, halo, nitro, cyano, NR^{a}R^{a}, NR^{a}S(O)₂R^{a}, NR^{a}C(O)R^{a}, NR^{a}CONR^{a}R^{a}, NR^{a}CO₂R^{a}, OR^{a}; SR^{a}, SOR^{a}, SO₃R^{a}, SO₂R^{a}, SO₂NR^{a}R^{a}, CO₂R^{a}, C(O)R^{a}, CONR^{a}R^{a}, CR^{a}R^{a}NR^{a}R^{a}, CR^{a}R^{a}OR^{a}, C₁-C₄-alkyl, C₂-C₄-alkenyl, C₂-C₄-alkynyl and C₁-C₄-haloalkyl; wherein R^{a} is independently at each occurrence selected from H, C₁-C₄ alkyl.

2. A compound of clause 1, wherein the compound of formula (I) is a compound of formula (IX):

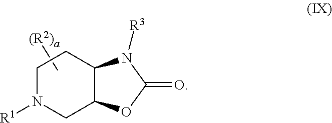

3. A compound of clause 1 or clause 2, wherein a is 0.

4. A compound of any preceding clause, wherein R¹ is:

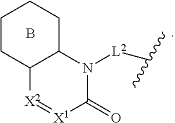

5. A compound of clause 4, wherein -L². is —C₂-alkylene-.

6. A compound of clause 4 or clause 5, wherein X¹ is CR⁴ and X² is CR⁵.

7. A compound of any one of clauses 4 to 5, wherein R⁴ and R⁵, together with the carbons to which they are attached together form a 5-membered heteroaryl ring.

8. A compound of clause 7, wherein the heteroaryl ring is a ring selected from oxazole, thiazole, isoxazole and isothiazole.

9. A compound of any one of clauses 4 to 8, wherein Ring B is a phenyl ring.

10. A compound of any one of clauses 4 to 8, wherein Ring B is a pyridine ring or a pyrimidine ring.

11. A compound of any one of clauses 1 to 3, wherein R¹ is -L¹-Ar¹-Ar²; wherein Ar¹ is independently selected from a phenyl or monocyclic heteroaryl group; and wherein Ar² is a monocyclic heteroaryl group.

12. A compound of clause 11, wherein -L¹. is —C₁-alkylene-.

13. A compound of clause 12, wherein Ar¹ is a phenyl group.

14. A compound of clause 12 or clause 13, wherein Ar² is a 6-membered heteroaryl group.

15. A compound of any preceding clause, wherein R³ takes the form:

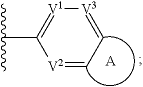

wherein V¹, V² and V³ are each independently selected from: N and CR¹⁰; with the proviso that no more than two of V¹, V² and V³ are N; and wherein the ring A is a substituted or unsubstituted 5- or 6-membered saturated cycloalkyl or heterocycloalkyl ring; and wherein R¹⁰ is independently at each occurrence selected from H, halo, nitro, cyano, NR^{a}R^{a}, NR^{a}S(O)₂R^{a}, NR^{a}C(O)R^{a}, NR^{a}CONR^{a}R^{a}, NR^{a}CO₂R^{a}, OR^{a}; SR^{a,} SOR^{a}, SO₃R^{a}, SO₂R^{a}, SO₂NR^{a}R^{a}, CO₂R^{a}, C(O)R^{a}, CONR^{a}R^{a}, CR^{a}R^{a}NR^{a}R^{a}, CR^{a}R^{a}OR^{a}, C₁-C₄-alkyl, C₂-C₄-alkenyl, C₂-C₄-alkynyl and C₁-C₄-haloalkyl.

### DETAILED DESCRIPTION

Throughout this specification, the term ‘compound of the invention’ is intended to refer to a compound of any one of formulae (I) to (IX) or a pharmaceutically acceptable salt or N-oxide thereof.

Where the compound of the invention is an N-oxide, it will typically be a pyridine N-oxide, i.e. where the compound of the invention comprises a pyridine ring (which may form part of a bicyclic or tricyclic ring system), the nitrogen of that pyridine may be N⁺—O⁻. Alternatively, it may be that the compound of the invention is not an N-oxide.

Included within the scope of the present invention are all stereoisomers, geometric isomers and tautomeric forms of the compounds of the invention, including compounds exhibiting more than one type of isomerism, and mixtures of one or more thereof. Also included are acid addition or base salts wherein the counter ion is optically active, for example, d-lactate or I-lysine, or racemic, for example, dl-tartrate or dl-arginine.

Compounds of the invention containing one or more asymmetric carbon atoms can exist as two or more stereoisomers. Where a compound of the invention contains a double bond such as a C═C or C═N group, geometric cis/trans (or Z/E) isomers are possible. Specifically, the oxime groups present in certain compounds of the invention may be present as the E-oxime, as the Z-oxime or as a mixture of both in any proportion. Cis/trans isomers may be separated by conventional techniques well known to those skilled in the art, for example, chromatography and fractional crystallisation.

Where structurally isomeric forms of a compound are interconvertible via a low energy barrier, tautomeric isomerism (‘tautomerism’) can occur. This can take the form of proton tautomerism in compounds of the invention containing, for example, an imino, keto, or oxime group, or so-called valence tautomerism in compounds which contain an aromatic moiety.

Conventional techniques for the preparation/isolation of individual enantiomers when necessary include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of the invention contains an acidic or basic moiety, a base or acid such as 1-phenylethylamine or tartaric acid. The resulting diastereomeric mixture may be separated by chromatography and/or fractional crystallization and one or both of the diastereoisomers converted into the corresponding pure enantiomer(s) by means well known to a skilled person.

Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and from 0 to 5% by volume of an alkylamine, typically 0.1% diethylamine. Concentration of the eluate affords the enriched mixture.

When any racemate crystallises, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer.

While both of the crystal forms present in a racemic mixture have identical physical properties, they may have different physical properties compared to the true racemate. Racemic mixtures may be separated by conventional techniques known to those skilled in the art—see, for example, “Stereochemistry of Organic Compounds” by E. L. Eliel and S. H. Wilen (Wiley, 1994).

It follows that a single compound may exhibit more than one type of isomerism.

The term Cₘ-Cₙ refers to a group with m to n carbon atoms.

The term “alkyl” refers to a monovalent linear or branched hydrocarbon chain. For example, C₁-C₆-alkyl may refer to methyl, ethyl, n-propyl, iso-propyl, n-butyl, sec-butyl, tert-butyl, n-pentyl and n-hexyl. An alkyl group may be unsubstituted or substituted by one or more substituents. Specific substituents for each alkyl group independently may be fluorine, OR^{a} or NHR^{a}.

The term “alkylene” refers to a bivalent linear hydrocarbon chain. For example, —C₁-C₃-alkyl may refer to methylene, ethylene or propylene. An alkylene group may be unsubstituted or substituted by one or more substituents. Specific substituents for each alkyl group independently may be methyl or ethyl.

The term “haloalkyl” refers to a hydrocarbon chain substituted with at least one halogen atom independently chosen at each occurrence from: fluorine, chlorine, bromine and iodine. The halogen atom may be present at any position on the hydrocarbon chain. For example, C₁-C₆-haloalkyl may refer to chloromethyl, fluoromethyl, trifluoromethyl, chloroethyl e.g. 1-chloromethyl and 2-chloroethyl, trichloroethyl e.g. 1,2,2-trichloroethyl, 2,2,2-trichloroethyl, fluoroethyl e.g. 1-fluoromethyl and 2-fluoroethyl, trifluoroethyl e.g. 1,2,2-trifluoroethyl and 2,2,2-trifluoroethyl, chloropropyl, trichloropropyl, fluoropropyl, trifluoropropyl. A halo alkyl group may be a fluoroalkyl group, i.e. a hydrocarbon chain substituted with at least one halogen atom.

The term “alkenyl” refers to a branched or linear hydrocarbon chain containing at least one double bond. The double bond(s) may be present as the E or Z isomer. The double bond may be at any possible position of the hydrocarbon chain. For example, “C₂-C₆-alkenyl” may refer to ethenyl, propenyl, butenyl, butadienyl, pentenyl, pentadienyl, hexenyl and hexadienyl. An alkenyl group may be unsubstituted or substituted by one or more substituents. Specific substituents for any saturated carbon atom in each alkenyl group independently may be fluorine, OR^{a} or NHR^{a}.

The term “alkynyl” refers to a branched or linear hydrocarbon chain containing at least one triple bond. The triple bond may be at any possible position of the hydrocarbon chain. For example, “C₂-C₆-alkynyl” may refer to ethynyl, propynyl, butynyl, pentynyl and hexynyl. An alkynyl group may be unsubstituted or substituted by one or more substituents. Specific substituents for any saturated carbon atom in each alkynyl group independently may be fluorine, OR^{a} or NHR^{a}.

The term “cycloalkyl” refers to a saturated hydrocarbon ring system containing 3, 4, 5 or 6 carbon atoms. For example, “C₃-C₆-cycloalkyl” may refer to cyclopropyl, cyclobutyl, cyclopentyl, cyclohexyl. A cycloalkyl group may be unsubstituted or substituted by one or more substituents. Specific substituents for each cycloalkyl group independently may be fluorine, OR^{a} or NHR^{a}.

The term “aromatic” when applied to a substituent as a whole means a single ring or polycyclic ring system with 4n+2 electrons in a conjugated π system within the ring or ring system where all atoms contributing to the conjugated π system are in the same plane.

The term “heteroaromatic” when applied to a substituent as a whole means a single ring or polycyclic ring system with 4n+2 electrons in a conjugated π system within the ring or ring system where all atoms contributing to the conjugated π system are in the same plane, the ring system comprising from 1 to 4 heteroatoms independently selected from O, S and N (in other words from 1 to 4 of the atoms forming the ring or ring system are selected from O, S and N).

The term “aryl” refers to an aromatic hydrocarbon ring system. The ring system has 4n+2 electrons in a conjugated π system within a ring where all atoms contributing to the conjugated π system are in the same plane. For example, the “aryl” may be phenyl and naphthyl. An aryl group may be unsubstituted or substituted by one or more substituents. Specific substituents for each aryl group independently may be C₁-C₄-alkyl, C₁-C₄-haloalkyl, cyano, halogen, OR^{a} or NHR^{a}.

Aryl groups may have from 6 to 20 carbon atoms as appropriate to satisfy valency requirements. Aryl groups comprise aromatic rings, i.e. rings which satisfy the Huckel rule. Aryl groups may be optionally substituted phenyl groups, optionally substituted biphenyl groups, optionally substituted naphthalenyl groups or optionally substituted anthracenyl groups. Equally, aryl groups may include non-aromatic carbocyclic portions. An aromatic ring is a phenyl ring.

The term “heteroaryl” may refer to any aromatic (i.e. a ring system containing (4n+2) π-electrons or n-electrons in the π-system) 5-10 membered ring system comprising from 1 to 4 heteroatoms independently selected from O, S and N (in other words from 1 to 4 of the atoms forming the ring system are selected from O, S and N). Thus, any heteroaryl groups may be independently selected from: 5 membered heteroaryl groups in which the heteroaromatic ring is substituted with 1-4 heteroatoms independently selected from O, S and N; and 6-membered heteroaryl groups in which the heteroaromatic ring is substituted with 1-3 (e.g. 1-2) nitrogen atoms; 9-membered bicyclic heteroaryl groups in which the heteroaromatic system is substituted with 1-4 heteroatoms independently selected from O, S and N; 10-membered bicyclic heteroaryl groups in which the heteroaromatic system is substituted with 1-4 nitrogen atoms. Specifically, heteroaryl groups may be independently selected from: pyrrole, furan, thiophene, pyrazole, imidazole, oxazole, isoxazole, triazole, oxadiazole, thiadiazole, tetrazole; pyridine, pyridazine, pyrimidine, pyrazine, triazine, indole, isoindole, benzofuran, isobenzofuran, benzothiophene, indazole, benzimidazole, benzoxazole, benzthiazole, benzisoxazole, purine, quinoline, isoquinoline, cinnoline, quinazoline, quinoxaline, pteridine, phthalazine, naphthyridine. Heteroaryl groups may also be 6-membered heteroaryl groups in which the heteroaromatic ring is substituted with 1 heteroatomic group independently selected from O, S and NH and the ring also comprises a carbonyl group. Such groups include pyridones and pyranones. The heteroaryl system itself may be substituted with other groups. A heteroaryl group may be unsubstituted or substituted by one or more substituents. Specific substituents for each heteroaryl group independently may be C₁-C₄-alkyl, C₁-C₄-haloalkyl, cyano, halogen, OR^{a} or NHR^{a}.

Heteroaryl groups may mean a 5- or 6-membered heteroaryl group. They may therefore comprise a 5- or 6-membered heteroaromatic ring, i.e. a 5- or 6-membered ring which satisfies the Huckel rule and comprises a heteroatom. Heteroaryl groups may be selected from: 5-membered heteroaryl groups in which the heteroaromatic ring is includes 1-4 heteroatoms selected from O, S and N; and 6-membered heteroaryl groups in which the heteroaromatic ring includes 1-2 nitrogen atoms. Specifically, heteroaryl groups and heteroaromatic rings may be selected from: pyrrole, furan, thiophene, pyrazole, imidazole, oxazole, isoxazole, triazole, oxadiazole, thiodiazole, pyridine, pyridazine, pyrimidine, pyrazine.

The term “_{y-z}-membered heterocycloalkyl” may refer to a monocyclic or bicyclic saturated or partially saturated group having from y to z atoms in the ring system and comprising 1 or 2 heteroatoms independently selected from O, S and N in the ring system (in other words 1 or 2 of the atoms forming the ring system are selected from O, S and N). By partially saturated it is meant that the ring may comprise one or two double bonds. This applies particularly to monocyclic rings with from 5 to 8 members. The double bond will typically be between two carbon atoms but may be between a carbon atom and a nitrogen atom. Examples of heterocycloalkyl groups include; piperidine, piperazine, morpholine, thiomorpholine, pyrrolidine, tetrahydrofuran, tetrahydrothiophene, dihydrofuran, tetrahydropyran, dihydropyran, dioxane, azepine. Bicyclic systems may be spiro-fused, i.e. where the rings are linked to each other through a single carbon atom; vicinally fused, i.e. where the rings are linked to each other through two adjacent carbon or nitrogen atoms; or they may be share a bridgehead, i.e. the rings are linked to each other two non-adjacent carbon or nitrogen atoms. A heterocycloalkyl group may be unsubstituted or substituted by one or more substituents. Specific substituents for any saturated carbon atom in each heterocycloalkyl group may independently be fluorine, OR^{a} or NHR^{a}.

An ‘endocyclic’ double bond is one where both of the atoms between which the double bond is formed are in the ring or ring system in which the atoms are situated.

A carbocyclic group consists of one or more rings which are entirely formed from carbon atoms. A carbocylic group can be a mono- or bicyclic cycloalkyl group, or it can comprise at least one phenyl ring.

A heterocyclic group consists of one or more rings wherein the ring system includes at least one heteroatom. A heterocyclic group comprises at least one heteroaryl or heterocycloalkyl rings. A heterocycloalkyl ring may be a saturated ring comprising at least one heteroatom selected from O, S and N.

Where a ring system is described as being a x-membered bicyclic group, that is intended to mean that the skeleton of the bicyclic ring system is formed from x atoms (i.e. the total number of atoms across the two rings of the bicycle is x).

Aryl and heteroaryl groups are optionally substituted with 1 to 5 substituents which are each independently at each occurrence selected from the group consisting of: halo, nitro, cyano, NR^{a}R^{a}, NR^{a}S(O)₂R^{a}, NR^{a}CONR^{a}R^{a}, NR^{a}CO₂R^{a}, NR^{a}C(O)R^{a}, OR^{a}; SR^{a,} SOR^{a}, SO₃R^{a}, SO₂R^{a}, SO₂NR^{a}R^{a}, CO₂R^{a} C(O)R^{a}, CONR^{a}R^{a}, C₁-C₄-alkyl, C₂-C₄-alkenyl, C₂-C₄-alkynyl, C₁-C₄-haloalkyl and CR^{a}R^{a}NR^{a}R^{a}; wherein R^{a} is independently at each occurrence selected from H, C₁-C₄-alkyl and C₁-C₄-haloalkyl.

The present invention also includes the synthesis of all pharmaceutically acceptable isotopically-labelled compounds of formulae (I) to (IX) wherein one or more atoms are replaced by atoms having the same atomic number, but an atomic mass or mass number different from the atomic mass or mass number most commonly found in nature. Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulfur, such as ³⁵S.

Certain isotopically-labelled compounds, for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labelled compounds can generally be prepared by conventional techniques known to those skilled in the art or by processes analogous to those described using an appropriate isotopically-labelled reagent in place of the non-labelled reagent previously employed.

### Uses, Methods of Treatment and Pharmaceutical Formulations

Each of the compounds of the present invention may be used as a medicament. Thus, in another aspect of the invention, there is provided a compound as defined above for the treatment of bacterial infections.

The compounds and formulations of the present invention may be used in the treatment of a wide range of bacterial infections. In some embodiments, the compounds can be used to treat bacterial infections caused by one or more resistant strains of bacteria e.g. a strain which is resistant to at least one approved antibiotic drug. In a further embodiment, the compounds can be used to treat bacterial infections caused by one or more resistant strains of Gram-positive bacteria e.g. a strain which is resistant to at least one approved antibiotic drug. In a further embodiment, the compounds can be used to treat bacterial infections caused by one or more resistant strains of Gram-negative bacteria, e.g. a strain which is resistant to at least one approved antibiotic drug.

The compounds and formulations of the invention may be used to treat infections caused by bacteria which are in the form of a biofilm.

The term ‘resistant strains’ is intended to mean strains of bacteria which have shown resistance to one or more known antibacterial drug. For example, it may refer to strains which are resistant to methicillin, strains that are resistant to one or more other β-lactam antibiotics, strains that are resistant to one or more fluoroquinolones and/or strains that are resistant to one or more other antibiotics (i.e. antibiotics other than β-lactams and fluoroquinolones). A resistant strain is one in which the MIC of a given compound or class of compounds for that strain has shifted to a significantly higher number than for the parent (susceptible) strain.

The term ‘approved drug’ is intended to mean that the drug is one which had been approved by the US FDA or the EMA prior to 1 Feb. 2016.

The bacterial strain (e.g. the MRSA strain or E. coli strain) may be resistant to one or more fluoroquinolone antibiotics, e.g. one or more antibiotics selected from levofloxacin, enoxacin, fleroxacin, lomefloxacin, nadifloxacin, norfloxacin, rufloxacin, balofloxacin, grepafloxacin, pazufloxacin, sparfloxacin, temafloxacin, tosufloxacin, besifloxacin, clinafloxacin, garenoxacin, gemifloxacin, gatifloxacin, sitafloxacin, trovafloxacin, prulifloxacin, ciprofloxacin, pefloxacin, moxifloxacin, ofloxacin, delafloxacin, zabofloxacin, avarofloxacin, finafloxacin.

The compounds of the invention may be particularly effective at treating infections caused by Gram-positive bacteria. The compounds of the invention may be particularly effective at treating infections caused by Gram-positive bacteria which are resistant to one or more fluoroquinolone antibiotics.

The compounds of the invention may be particularly effective at treating infections caused by Gram-negative bacteria. The compounds of the invention may be particularly effective at treating infections caused by Gram-negative bacteria which are resistant to one or more fluoroquinolone antibiotics.

The compounds of the invention may be particularly effective at treating infections caused by aerobic bacteria, e.g. S. aureus. The compounds of the invention may be particularly effective at treating infections caused by anaerobic bacteria, e.g. a Clostridium spp. such as Clostridium difficile.

The compounds and formulations of the present invention can be used to treat or to prevent infections caused by bacterial strains associated with biowarfare. These may be strains which are category A pathogens as identified by the US government (e.g. those which cause anthrax, plague etc.) and/or they may be strains which are category B pathogens as identified by the US government (e.g. those which cause Glanders disease, mellioidosis etc). In a specific embodiment, the compounds and formulations of the present invention can be used to treat or to prevent infections caused by Gram-positive bacterial strains associated with biowarfare (e.g. anthrax). More particularly, the compounds and formulations may be used to treat category A and/or category B pathogens as defined by the US government on 1 Jan. 2014.

The bacterial infection may be caused by a strain selected from: Neisseria spp., Haemophilus spp., Legionella spp., Pasteurella spp., Bordetella spp., Brucella spp., Francisella spp. and Moraxella spp. Like Neisseria spp., Haemophilus spp., Legionella spp., Pasteurella spp., Bordetella spp., Brucella spp., Francisella spp. and Moraxella spp. are fastidious Gram-negative organisms. A fastidious bacterium is one having a complex nutritional requirement, i.e. one which will only grow when specific nutrients are included in the culture medium. As an example Neisseria gonorrhoeae requires, amongst other supplements, iron, several amino acids, cofactors and vitamins in order to grow. Members of the fastidious Gram-negative bacteria group often share common antibiotic susceptibility profiles. Pathogenic Neisseria species include Neisseria gonorrhoeae (the pathogen responsible for gonorrhoea) and Neisseria meningitidis (one of the pathogens responsible for bacterial meningitis). Infections which can be treated by the compounds and methods of the invention include gonorrhoea. Infections which can be treated include secondary infections which can arise from lack of treatment of a primary Neisseria gonorrhoeae infection. Exemplary secondary infections include urethritis, dysuria, epididymitis, pelvic inflammatory disease, cervicitis and endometritis and also systemic gonococcal infections (e.g. those manifesting as arthritis, endocarditis or meningitis). The gonorrhoea infection may be one caused by a strain of Neisseria gonorrhoeae which is resistant to at least one known antibacterial drug, e.g. at least one β-lactam drug. The gonorrhoea infection may be one caused by a strain of Neisseria gonorrhoeae which is resistant to at least one approved drug. The at least one drug may be an antibiotic drug, e.g. one that is approved for use in treating one of the fastidious Gram-negative species mentioned in this specification. It may be approved for use in treating gonorrhoea. The approved drug may be a β-lactam drug. Further infections which can be treated by the compounds and methods of the invention include bacterial meningitis and Neisseria meningitidis infections of other parts of the human or animal body.

The compounds of the invention can be used to treat or prevent mycobacterial infections, e.g. mycobacterial infections caused by resistant strains of mycobacteria. Thus, for example, they can be used to treat TB or leprosy. Thus, it may be that the mycobacterial infection is caused by M. tuberculosis. It may also be that the mycobacterial infection is caused by a mycobacterium selected from: M. avium complex, M. abscessus, M. leprae, M. bovis, M. kansasii, M. chelonae, M. africanum, M. canetti and M. microti. The compounds may be used to treat resistant strains of TB, e.g. MDR-TB (i.e. TB infections caused by strains which are resistant to isoniazid and rifampicin), XDR-TB (i.e. TB infections caused by strains which are resistant to isoniazid, rifampicin, at least one fluoroquinolone and at least one of kanamycin, capreomycin and amikacin) and/or TDR-TB (i.e. TB infections caused by strains which have proved resistant to every drug tested against it with the exception of a compound of the invention). The mycobacterium is caused by a mycobacterial strain which is resistant to at least one approved antimycobacterial compound. The at least one approved antimycobacterial compound may be selected from: rifampicin, isoniazid, kanamycin, capreomycin, amikacin and a fluoroquinolone. The at least one approved antimycobacterial compound may be selected from: rifampicin, moxifloxacin, isoniazid, ciprofloxacin and levofloxacin. The compounds of the invention may be used to treat non-replicating TB.

The compounds of the invention may also be useful in treating other forms of infectious disease, e.g. fungal infections, parasitic infections and/or viral infections.

The compounds of the present invention can be used in the treatment of the human body. They may be used in the treatment of the animal body. In particular, the compounds of the present invention can be used to treat commercial animals such as livestock. Alternatively, the compounds of the present invention can be used to treat companion animals such as cats, dogs, etc.

The compounds of the invention may be obtained, stored and/or administered in the form of a pharmaceutically acceptable salt. Suitable pharmaceutically acceptable salts include, but are not limited to, salts of pharmaceutically acceptable inorganic acids such as hydrochloric, sulfuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, malic, citric, lactic, mucic, gluconic, benzoic, succinic, oxalic, phenylacetic, methanesulfonic, toluenesulfonic, benzenesulfonic, salicylic, sulfanilic, aspartic, glutamic, edetic, stearic, palmitic, oleic, lauric, pantothenic, tannic, ascorbic and valeric acids. Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulfate and hemicalcium salts. Also included are acid addition or base salts wherein the counter ion is optically active, for example, d-lactate or I-lysine, or racemic, for example, dl-tartrate or dl-arginine.

Compounds of the invention may exist in a single crystal form or in a mixture of crystal forms or they may be amorphous. Thus, compounds of the invention intended for pharmaceutical use may be administered as crystalline or amorphous products. They may be obtained, for example, as solid plugs, powders, or films by methods such as precipitation, crystallization, freeze drying, or spray drying, or evaporative drying. Microwave or radio frequency drying may be used for this purpose.

For the above-mentioned compounds of the invention the dosage administered will, of course, vary with the compound employed, the mode of administration, the treatment desired and the disorder indicated. For example, if the compound of the invention is administered orally, then the daily dosage of the compound of the invention may be in the range from 0.01 micrograms per kilogram body weight (μg/kg) to 100 milligrams per kilogram body weight (mg/kg).

A compound of the invention, or pharmaceutically acceptable salt thereof, may be used on their own but will generally be administered in the form of a pharmaceutical composition in which the compounds of the invention, or pharmaceutically acceptable salt thereof, is in association with a pharmaceutically acceptable adjuvant, diluent or carrier. Conventional procedures for the selection and preparation of suitable pharmaceutical formulations are described in, for example, “Pharmaceuticals—The Science of Dosage Form Designs”, M. E. Aulton, Churchill Livingstone, 1988.

The compounds of the invention may be administered in combination with other active compounds (e.g. antifungal compounds, oncology compounds) and, in particular, with other antibacterial compounds. The compound of the invention and the other active (e.g. the other antibacterial compound) may be administered in different pharmaceutical formulations either simultaneously or sequentially with the other active. Alternatively, the compound of the invention and the other active (e.g. the other antibacterial compound) may form part of the same pharmaceutical formulation.

Examples of other bacterial compounds which could be administered with the compounds of the invention are penems, carbapenems, fluoroquinolones, β-lactams, vancomycin, erythromycin or any other known antibiotic drug molecule.

Depending on the mode of administration of the compounds of the invention, the pharmaceutical composition which is used to administer the compounds of the invention will preferably comprise from 0.05 to 99% w (percent by weight) compounds of the invention, more preferably from 0.05 to 80% w compounds of the invention, still more preferably from 0.10 to 70% w compounds of the invention, and even more preferably from 0.10 to 50% w compounds of the invention, all percentages by weight being based on total composition.

The pharmaceutical compositions may be administered topically (e.g. to the skin) in the form, e.g., of creams, gels, lotions, solutions, suspensions, or systemically, e.g. by oral administration in the form of tablets, capsules, syrups, powders, suspensions, solutions or granules; or by parenteral administration in the form of a sterile solution, suspension or emulsion for injection (including intravenous, subcutaneous, intramuscular, intravascular or infusion); or by rectal administration in the form of suppositories; or by inhalation (i.e. in the form of an aerosol or by nebulisation).

If administered topically, high-dosages of the compounds of the invention can be administered. Thus, a compound with an in vitro MIC of, for example, 16-64 μg/mL may still provide an effective treatment against certain bacterial infections.

For oral administration the compounds of the invention may be admixed with an adjuvant or a carrier, for example, lactose, saccharose, sorbitol, mannitol; a starch, for example, potato starch, corn starch or amylopectin; a cellulose derivative; a binder, for example, gelatine or polyvinylpyrrolidone; and/or a lubricant, for example, magnesium stearate, calcium stearate, polyethylene glycol, a wax, paraffin, and the like, and then compressed into tablets. If coated tablets are required, the cores, prepared as described above, may be coated with a concentrated sugar solution which may contain, for example, gum arabic, gelatine, talcum and titanium dioxide. Alternatively, the tablet may be coated with a suitable polymer dissolved in a readily volatile organic solvent.

For the preparation of soft gelatine capsules, the compounds of the invention may be admixed with, for example, a vegetable oil or polyethylene glycol. Hard gelatine capsules may contain granules of the compound using either the above-mentioned excipients for tablets. Also liquid or semisolid formulations of the compound of the invention may be filled into hard gelatine capsules. Liquid preparations for oral application may be in the form of syrups or suspensions, for example, solutions containing the compound of the invention, the balance being sugar and a mixture of ethanol, water, glycerol and propylene glycol. Optionally such liquid preparations may contain colouring agents, flavouring agents, sweetening agents (such as saccharine), preservative agents and/or carboxymethylcellulose as a thickening agent or other excipients known to those skilled in the art.

For intravenous (parenteral) administration the compounds of the invention may be administered as a sterile aqueous or oily solution.

The size of the dose for therapeutic purposes of compounds of the invention will naturally vary according to the nature and severity of the conditions, the age and sex of the animal or patient and the route of administration, according to well known principles of medicine.

Dosage levels, dose frequency, and treatment durations of compounds of the invention are expected to differ depending on the formulation and clinical indication, age, and co-morbid medical conditions of the patient. The standard duration of treatment with compounds of the invention is expected to vary between one and seven days for most clinical indications. It may be necessary to extend the duration of treatment beyond seven days in instances of recurrent infections or infections associated with tissues or implanted materials to which there is poor blood supply including bones/joints, respiratory tract, endocardium, and dental tissues.

In another aspect the present invention provides a pharmaceutical formulation comprising a compound of the invention and a pharmaceutically acceptable excipient. The formulation may further comprise one or more other antibiotics, e.g. one or more fluoroquinolone antibiotics. Illustrative fluoroquinolone antibiotics include levofloxacin, enoxacin, fleroxacin, lomefloxacin, nadifloxacin, norfloxacin, rufloxacin, balofloxacin, grepafloxacin, pazufloxacin, sparfloxacin, temafloxacin, tosufloxacin, besifloxacin, clinafloxacin, garenoxacin, gemifloxacin, gatifloxacin, sitafloxacin, trovafloxacin, prulifloxacin, ciprofloxacin, pefloxacin, moxifloxacin, ofloxacin, delafloxacin, zabofloxacin, avarofloxacin, finafloxacin.

In another aspect of the invention is provided a method of treating a bacterial infection, the method comprising treating a subject in need thereof with a therapeutically effective amount of a compound of the invention.

### Medical Uses

The compounds of the present invention can be used in the treatment of the human body.

The compounds of the invention may be for use in treating human bacterial infections such as infections of the genitourinary system, the respiratory tract, the gastrointestinal tract, the ear, the skin, the throat, soft tissue, bone and joints (including infections caused by Staphylococcus aureus). The compounds can be used to treat pneumonia, sinusitis, acute bacterial sinusitis, bronchitis, acute bacterial exacerbation of chronic bronchitis, anthrax, chronic bacterial prostatitis, acute pyelonephritis, pharyngitis, tuberculosis, tonsillitis, Escherichia coli, prophylaxis before dental surgery, cellulitis, acnes, cystitis, infectious diarrhoea, typhoid fever, infections caused by anaerobic bacteria, peritonitis, abdominal infection, bacteraemia, septicaemia, sexually transmitted bacterial infection (e.g. gonorrhoea, Chlamydia), bacterial vaginosis, pelvic inflammatory disease, pseudomembranous colitis, Helicobacter pylori, acute gingivitis, Crohn's disease, rosacea, fungating tumours, impetigo.

The compounds of the present invention may also be used in treating other conditions treatable by eliminating or reducing a bacterial infection. In this case they will act in a secondary manner alongside for example a chemotherapeutic agent used in the treatment of cancer.

In yet another aspect of the invention is provided a compound for use in the preparation of a medicament. The medicament may be for use in the treatment of any of the diseases, infections and indications mentioned in this specification.

In an aspect of the invention is provided a compound of the invention for medical use. The compound may be used in the treatment of any of the diseases, infections and indications mentioned in this specification.

### Veterinary Uses

They may be used in the treatment of the animal body. In particular, the compounds of the present invention can be used to treat commercial animals such as livestock. The livestock may be mammal (excluding humans) e.g. cows, pigs, goats, sheep, llamas, alpacas, camels and rabbits. The livestock may be birds (e.g. chickens, turkeys, ducks, geese etc.). Alternatively, the compounds of the present invention can be used to treat companion animals such as cats, dogs, etc. The veterinary use may be to treat wild populations of animals in order to prevent the spread of disease to humans or to commercial animals. In this case, the animals may be rats, badgers, deer, foxes, wolves, mice, kangaroos and monkeys and other apes.

In an aspect of the invention is provided a compound of the invention for veterinary use. The compound may be used in the treatment of any of the animal diseases and infections and indications mentioned in this specification.

In another aspect the present invention provides a veterinary formulation comprising a compound of the invention and a veterinarily acceptable excipient.

The methods by which the compounds may be administered for veterinary use include oral administration by capsule, bolus, tablet or drench, topical administration as an ointment, a pour-on, spot-on, dip, spray, mousse, shampoo, collar or powder formulation or, alternatively, they can be administered by injection (e.g. subcutaneously, intramuscularly or intravenously), or as an implant. Such formulations may be prepared in a conventional manner in accordance with standard veterinary practice. The formulations will vary with regard to the weight of active compound contained therein, depending on the species of animal to be treated, the severity and type of infection and the body weight of the animal. For parenteral, topical and oral administration, typical dose ranges of the active ingredient are 0.01 to 100 mg per kg of body weight of the animal. Preferably the range is 0.1 to 10 mg per kg. In any event, the veterinary practitioner, or the skilled person, will be able to determine the actual dosage which will be most suitable for an individual patient, which may vary with the species, age, weight and response of the particular patient. The above dosages are exemplary of the average case; there can, of course, be individual instances where higher or lower dosage ranges are merited, and such are within the scope of this invention.

As an alternative, when treating animals the compounds may be administered with the animal feedstuff and for this purpose a concentrated feed additive or premix may be prepared for mixing with the normal animal feed.

Certain compounds of the invention are of particular use in the treatment of mastitis. In this regard, a particularly preferred method of administration is by injection into the udder of a subject (e.g. a cow, a goat, a pig or sheep).

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

### Synthesis

The skilled man will appreciate that adaptation of methods known in the art could be applied in the manufacture of the compounds of the present invention.

For example, the skilled person will be immediately familiar with standard textbooks such as “Comprehensive Organic Transformations—A Guide to Functional Group Transformations”, R C Larock, Wiley-VCH (1999 or later editions), “March's Advanced Organic Chemistry—Reactions, Mechanisms and Structure”, MB Smith, J. March, Wiley, (5th edition or later) “Advanced Organic Chemistry, Part B, Reactions and Synthesis”, F A Carey, R J Sundberg, Kluwer Academic/Plenum Publications, (2001 or later editions), “Organic Synthesis—The Disconnection Approach”, S Warren (Wiley), (1982 or later editions), “Designing Organic Syntheses” S Warren (Wiley) (1983 or later editions), “Guidebook To Organic Synthesis” R K Mackie and DM Smith (Longman) (1982 or later editions), etc., and the references therein as a guide.

The skilled chemist will exercise his judgement and skill as to the most efficient sequence of reactions for synthesis of a given target compound and will employ protecting groups as necessary. This will depend inter alia on factors such as the nature of other functional groups present in a particular substrate. Clearly, the type of chemistry involved will influence the choice of reagent that is used in the said synthetic steps, the need, and type, of protecting groups that are employed, and the sequence for accomplishing the protection/deprotection steps. These and other reaction parameters will be evident to the skilled person by reference to standard textbooks and to the examples provided herein.

Sensitive functional groups may need to be protected and deprotected during synthesis of a compound of the invention. This may be achieved by conventional methods, for example as described in “Protective Groups in Organic Synthesis” by TW Greene and PGM Wuts, John Wiley & Sons Inc (1999), and references therein.

Throughout this specification these abbreviations have the following meanings:

ACN—Acetonitrile Cbz—benzyl carbonate
CDI—Carbonyl diimidazole BOC—tert-Butyl carbonate
dba—Dibenzylideneacetone DCE—1,2-dichloroethane

### DCM—Dichloromethane DMF—N,N-Dimethylformamide

DMSO—Dimethylsulfoxide IPA—iso-Propyl alcohol
mCPBA—meta-chloroperbenzoic acid NMP—N-Methylpyrolidinone
PM B—para-Methoxybenzyl TFA—Trifluoroacetic acid

### THF—Tetrahydrofuran

Certain compounds of the invention can be made according to the following general schemes. Certain compounds of the invention can be made according to or analogously to the methods described in Examples 1 to 10

Certain compounds of formula (I) can be made by Scheme A:—

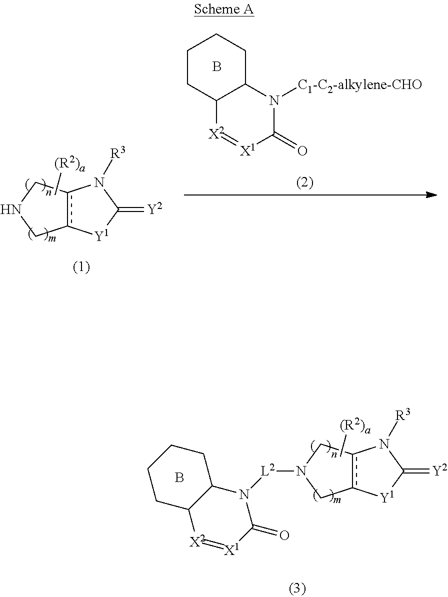

Amine (1) can be converted to (3) (a subset of compounds of formula (I)) via reductive amination with aldehyde (2). The reaction can be performed using a borohydride reagent, such as tetramethylammonium triacetoxyborohydride or sodium triacetoxyborohydride, in a solvent, such as THF or 1,2-dichloroethane, at a temperature from room temperature to 80° C. Addition of 4 Å sieves is optional.

Following Scheme A but using aldehyde (4), compound (5) (a subset of compounds of formula (I)) can be prepared.

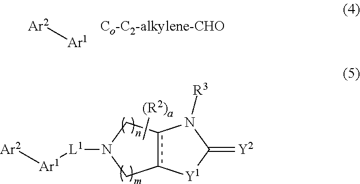

Following Scheme A but using amine (6), compound (7) (a subset of compounds of formula (III)) can be prepared.

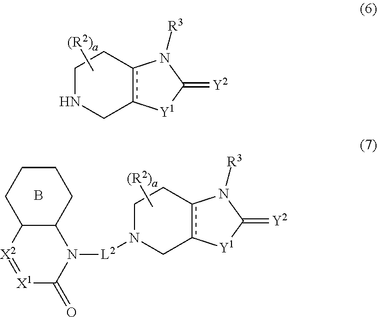

Following Scheme A but using amine (6) and aldehyde (4), compound (8) (a subset of compounds of formula (III)) can be prepared.

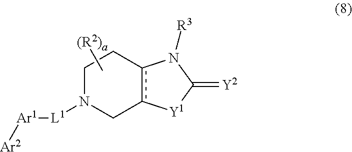

Following Scheme A but using amine (9) and aldehyde (4), compound (10) (a subset of compounds of formula (V)) can be prepared.

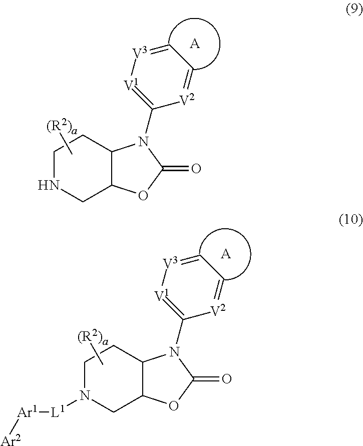

Following Scheme A but using amine (9) and aldehyde (2), compound (11) (a subset of compounds of formula (V)) can be prepared.

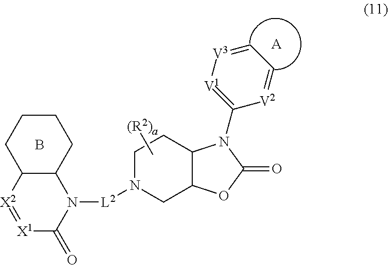

Following Scheme A, but using aldehyde (12) and amine (13), compound (14) (a subset of compounds of formula (VI)) can be prepared.

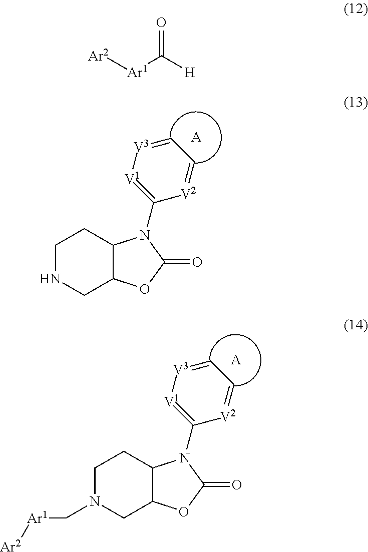

Following Scheme A, but using aldehyde (15) and amine (13), compound (16) (a subset of compounds of formula (VII)) can be prepared.

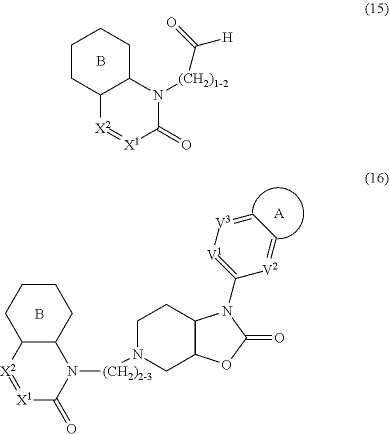

Aldehyde (15) can be made by Scheme B:—

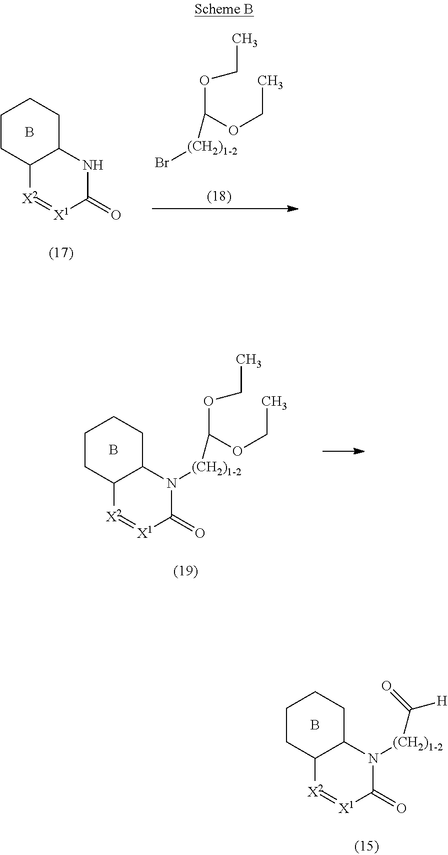

Reaction of pyridone (17) with commercially available bromo acetals (18) can generate pyridone acetals (19). The alkylation reaction can be carried out in the presence of a base, such as Cs₂CO₃, in a solvent, such as dry NMP, at a temperature from 50-100° C. Hydrolysis of pyridone acetals (19) to give the requisite aldehyde (15) can be effected using a strong acid, such as concentrated HCl, in a solvent, such as ACN, at room temperature.

Amine (13) can be made by Scheme C:—

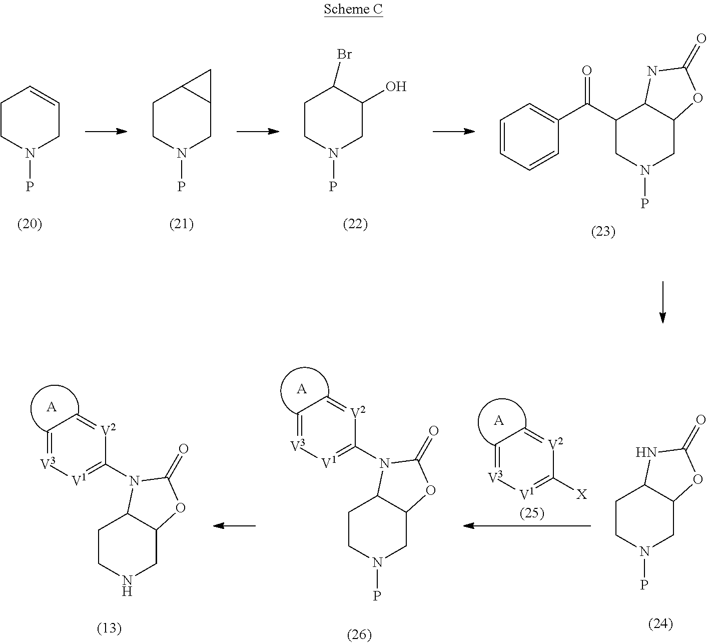

Reaction of protected amine (20), where P represents a standard nitrogen protecting group, such as Cbz, with an epoxidising reagent, such as m-CPBA, in a solvent, such as DCM, at a temperature from 0° C. to room temperature can generate epoxide (21). Ring opening of the epoxide under the influence of aqueous HBr can generate bromohydrin (22). The reaction can be performed in DCM at a temperature of −40° C. to 0° C. The bromohydrin (22) can be converted to (23) via a two-step process involving initial reaction with benzoyl isocyanate in a solvent, such as THF, at a temperature from 0° C. to room temperature, followed by addition of a base, such as t-BuOK or NaH and maintaining a reaction temperature from 0° C. to 65° C. Hydrolysis of the benzoyl group to generate oxazolidinone (24) can be affected by base, such as LiOH, or acid, such as HCl, in a solvent mixture of H₂O/THF at a temperature from room temperature to 70° C. Cross coupling reaction of (24), with (25), where X═Br or Cl, can generate protected amine (26). The cross coupling reaction can be copper catalysed, using for example CuI, in the presence of a diamine, such as 1,2-diaminocyclohexane or N,N-dimethyl-1,2-ethanediamine, in the presence of a base, such as K₂CO₃, in a solvent, such as dioxane or toluene, at a temperature from 70-110° C. The cross coupling reaction can be palladium catalysed, using for example, Pd₂(dba)₃ or Pd(OAc)₂ in the presence of a phosphine, such as Xantphos or P(t-Bu)₃ or X-Phos, in the presence of a base, such as t-BuONa or Cs₂CO₃, in a solvent, such as dioxane or toluene, at a temperature from 80-120° C. The nitrogen protecting group in (26) can be deprotected to give the free amine (13) under standard conditions. Where the nitrogen protecting group is Cbz the deprotection can be achieved by the action of H₂ in the presence of Pd/C in an alcoholic solvent, such as EtOH, at room temperature.

Following Scheme B, but using tricyclic pyridone (27), tricyclic aldehyde (28) can be prepared.

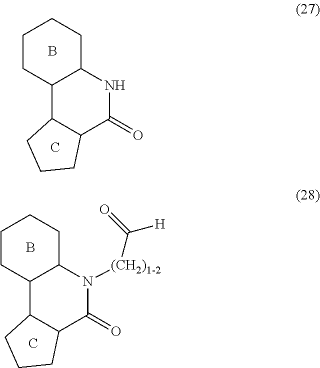

Following Scheme A, but using aldehyde (28) and amine (13), compound (29) (a subset of compounds of formula (VIII)) can be prepared.

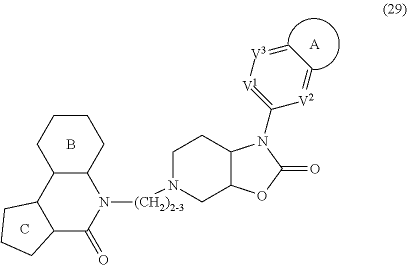

### Experimental

### Analytical Methods

NMR spectra were obtained on a LC Bruker AV400 using a 5 mm QNP probe (Method A), a Bruker AVIII 400 Nanobay using a 5 mm BBFQ with z-gradients (Method B), a Bruker AV1 Avance using a 5 mm QNP probe (Method C), a Bruker AV1 Avance using a 1H/13C Dual probe (Method D) or a Bruker ASCEND 400 MHz spectrometer (Method E).

MS was carried out on either a Waters ZQ MS (Method A and B), an Agilent Technologies 1200 series (Method C and D) using H₂O and ACN (0.1% formic acid—acidic pH; 0.1% ammonia—basic pH)—wavelengths were 254 and 210 nM, or a Shimadzu LCMS-2020 (Method E) using H₂O and MeOH (0.1% formic acid—acidic pH; 0.1% ammonia—basic pH)—wavelengths were 254 and 210 nm.

### Method A

Column: YMC-Triart C18, 5 μm, 50×2 mm. Flow rate: 0.8 mL/min. Injection volume 5 μL.

| Time | H₂O | ACN |
|---|---|---|
| (min) | % | % |
| 0 | 95 | 0 |
| 4 | 0 | 95 |
| 4.4 | 0 | 95 |
| 4.5 | 95 | 5 |

| | |
|---|---|
| 4.5 | STOP |

### Method B

Column: YMC-Triart C18, 5 μm, 50×2 mm. Flow rate: 0.8 mL/min. Injection volume 5-10 μL

| Time (min) | H₂O (%) | ACN (%) |
|---|---|---|
| 0 | 95 | 0 |
| 2.0 | 95 | 0 |
| 12.0 | 0 | 95 |
| 14.0 | 0 | 95 |
| 14.2 | 95 | 0 |

### Method C

Column: Poroshell 120, 2.7 μm, 50×2.1 mm. Flow rate: 0.8 mL/min. Injection volume 5-10 μL

| Time | H₂O | ACN |
|---|---|---|
| (min) | (%) | (%) |
| 0.0 | 90 | 10 |
| 0.5 | 90 | 10 |
| 4.0 | 20 | 80 |
| 5.0 | 20 | 80 |
| 5.1 | 90 | 10 |
| 6.0 | 90 | 10 |

### Method D

Column: Poroshell 120, 2.7 μm, 50×2.1 mm. Flow rate: 0.8 mL/min. Injection volume 5-10 μL

| Time | H₂O | ACN |
|---|---|---|
| (min) | (%) | (%) |
| 0.0 | 90 | 10 |
| 0.5 | 90 | 10 |
| 5.0 | 20 | 80 |
| 6.0 | 20 | 80 |
| 6.1 | 90 | 10 |
| 7.0 | 90 | 10 |

### Method E

Column: Atlantis T3, 3 μm, 3.0×75 mm. Flow rate: 0.8 mL/min. Injection volume 1-10 μL

| Time (min) | H₂O (%) | MeOH (%) |
|---|---|---|
| 0.01 | 50 | 50 |
| 0.05 | 50 | 50 |
| 3.5 | 95 | 5 |
| 7.0 | 95 | 5 |
| 8.5 | 50 | 50 |
| 9.5 | 50 | 50 |

Preparative HPLC was performed using a Waters 3100 Mass detector (Method A) or Waters 2767 Sample Manager (Method B) using H₂O and ACN (0.1-% formic acid—acidic pH; 0.1% ammonia—basic pH).

### Method A

Column: XBridge™ prep C18, 5 μm OBD 19×100 mm. Flow rate: 20 mL/min.

### Method B

Column: XBridge™ prep C18, 5 μm OBD 19×100 mm. Flow rate: 20 mL/min.

### Example 1:—rel-1-{2-[(3aR*,7aS*)-2-oxo-1-{3-oxo-2H,3H,4H-pyrido[3,2-b][1,4]oxazin-6-yl}-octahydro-[1,3]oxazolo[5,4-c]pyridin-5-yl]ethyl}-7-methoxy-1,2-dihydro-1,8-naphthyridin-2-one

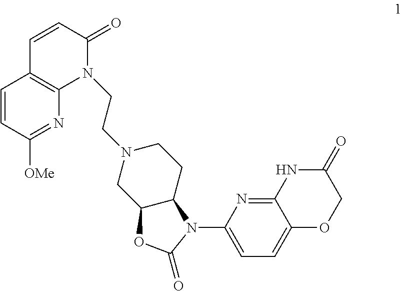 [SMILES: COc1ccc2ccc(=O)n(CCN3CC[C@@H]4[C@H](C3)OC(=O)N4c3ccc4c(n3)NC(=O)CO4)c2n1]

### a) rac-benzyl 7-oxa-3-azabicyclo[4.1.0]heptane-3-carboxylate 1a

To a solution of benzyl 1,2,3,6-tetrahydropyridine-1-carboxylate (71.4 g, 0.33 mol) in DCM (400 mL) at room temperature was added m-CPBA (80%) (85.1 g, 0.39 mol) in small portions and the mixture was stirred at room temperature for 2 h. The mixture was diluted with a saturated aqueous Na₂SO₃ solution, the layers were separated and the organic phase was washed with H₂O (500 mL×2), brine (300 mL), dried over Na₂SO₄ and concentrated. The residue was purified by silica gel chromatography (10% EtOAc in petroleum ether) to give rac-benzyl 7-oxa-3-azabicyclo[4.1.0]heptane-3-carboxylate 1a (63.1 g, 82%) as a yellow oil. TLC:R_{f}=0.15 (silica gel, petroleum ether/EtOAc=5:1, v/v).

¹H NMR (Method E) (CDCl₃): δ ppm 7.41-7.27 (m, 5H), 5.12 (s, 2H), 3.97 (m, 1H), 3.78 (m, 1H), 3.52 (m, 1H), 3.30-3.19 (m, 3H), 2.08 (m, 1H), 1.94 (m, 1H).

### b) rel-benzyl (3R*,4R*)-4-bromo-3-hydroxypiperidine-1-carboxylate 1b

To a solution of rac-benzyl 7-oxa-3-azabicyclo[4.1.0]heptane-3-carboxylate 1a (63.1 g, 0.27 mol) in DCM (800 mL) at -400C under N₂ was added 40% HBr (140 mL) dropwise and the mixture was stirred at −40° C. for 5 h. The mixture was washed with H₂O (800 mL×2), 10% aqueous NaHCO₃ (300 mL×2), brine (400 mL), dried over Na₂SO₄ and concentrated to give rel-benzyl (3R*,4R*)-4-bromo-3-hydroxypiperidine-1-carboxylate 1b (81.4 g, 96%) as a yellow oil. TLC:R_{f}=0.35 (silica gel, petroleum.ether/EtOAc=2:1, v/v). LC-MS (Method C) 315.1 [M+H]⁺; RT 3.64 min. NMR (Method E) (CDCl₃): δ ppm 7.34-7.22 (m, 5H), 5.06 (s, 2H), 4.20 (m, 1H), 4.01-3.79 (m, 2H), 3.65 (m, 1H), 3.00 (m, 1H), 2.92 (d, J=12.1 Hz, 1H), 2.46 (m, 1H), 2.29-2.21 (m, 1H), 1.92 (m, 1H).

### c) rel-benzyl (3aR*,7aS*)-1-benzoyl-2-oxo-octahydro-[1,3]oxazolo[5,4-c]pyridine-5-carboxylate 1c

To a solution of rel-benzyl (3R*,4R*)-4-bromo-3-hydroxypiperidine-1-carboxylate 1b (79.0 g, 0.25 mol) in THF (100 mL) at room temperature under N₂ was added benzoyl isocyanate (48.2 g, 0.33 mol) and the mixture was stirred at room temperature for 1 h to give int 1c (TLC:R_{f}=0.3 (silica gel, DCM/MeOH=20:1, v/v), LC-MS (Method C) 461.1 [M+H]⁺; RT 4.20 min). The mixture was diluted with THF (600 mL) and t-BuOK (28.3 g, 0.25 mmol) added. The mixture was heated at reflux for 1 h and allowed to cool then diluted with EtOAc (800 mL), washed with H₂O (800 mL×3), brine (400 mL), dried over Na₂SO₄ and concentrated. The residue was purified by silica gel chromatography (10-50% EtOAc in petroleum ether) to give rel-benzyl (3aR*,7aS*)-1-benzoyl-2-oxo-octahydro-[1,3]oxazolo[5,4-c]pyridine-5-carboxylate 1c (78.0 g, 82%) as a colorless oil. TLC:R_{f}=0.1 (silica gel, petroleum.ether/EtOAc=20:1, v/v). LC-MS (Method C) 381.1 [M+H]⁺; RT 3.95 min.

### d) rel-benzyl (3aR*,7aS*)-2-oxo-octahydro-[1,3]oxazolo[5,4-c]pyridine-5-carboxylate d

A mixture of rel-benzyl (3aR*,7aS*)-1-benzoyl-2-oxo-octahydro-[1,3]oxazolo[5,4-c]pyridine-5-carboxylate 1c (80.5 g, 0.21 mmol) and LiOH.H₂O (8.90 g, 0.21 mmol) in THF (600 mL) and H₂O (150 mL) was stirred at room temperature for 1 h. The mixture was diluted with H₂O (1 L), extracted with EtOAc (500 mL×2) and the combined extracts were washed with H₂O (1 L×2), brine (400 mL), dried over Na₂SO₄ and concentrated to give the crude product (61.6 g, >100%) as a yellow oil. A portion (10.0 g) was purified by silica gel chromatography (5% MeOH in DCM) to give pure rel-benzyl (3aR*,7aS*)-2-oxo-octahydro-[1,3]oxazolo[5,4-c]pyridine-5-carboxylate Id (5.10 g) as a white solid. TLC:R_{f}=0.1 (silica gel, DCM/MeOH=20:1, v/v). LC-MS (Method C) 277.1 [M+H]⁺; RT 2.80 min. NMR (Method E) (CDCl₃): δ ppm 7.42-7.27 (m, 5H), 6.32 (br s, 1H), 5.17 (m, 2H), 4.78 (m, 1H), 4.07 (m, 2H), 3.66-3.36 (m, 3H), 1.99-1.70 (m, 2H).

### e) rel-benzyl (3aR*,7aS*)-1-{4-[(4-methoxyphenyl)methyl]-3-oxo-2H,3H,4H-pyrido[3,2-b][1,4]oxazin-6-yl}-2-oxo-octahydro-[1,3]oxazolo[5,4-c]pyridine-5-carboxylate 1e

Trans-1,2-diaminocyclohexane (1.38 mL) was added dropwise to a solution of 6-bromo-4-[(4-methoxyphenyl)methyl]-2H,3H,4H-pyrido[3,2-b][1,4]oxazin-3-one (prepared as described in WO2014108832) (2.21 g, 6.33 mmol), CuI (242 mg, 1.27 mmol), K₂CO₃ (1.75 g, 12.66 mmol) and rel-benzyl (3aR*,7aS*)-2-oxo-octahydro-[1,3]oxazolo[5,4-c]pyridine-5-carboxylate Id (1.75 g, 6.33 mmol) in dioxane (100 mL) at 0° C. under N₂ and the resulting mixture was then heated at 100° C. for 24 h. After cooling to room temperature, the mixture was diluted with water and exacted with EtOAc (500 mL). The organic layer was washed with brine, dried over Na₂SO₄, filtered and concentrated under reduced pressure. The residue was purified by silica gel chromatography (20% EtOAc in petroleum ether then 2% MeOH in DCM) to give rel-benzyl (3aR*,7aS*)-1-{4-[(4-methoxyphenyl)methyl]-3-oxo-2H,3H,4H-pyrido[3,2-b][1,4]oxazin-6-yl}-2-oxo-octahydro-[1,3]oxazolo[5,4-c]pyridine-5-carboxylate 1e (3.35 g, 97%) as a solid. TLC:R_{f}=0.3 (silica gel, DCM/MeOH=50:1, v/v). LC-MS (Method C) 545.2 [M+H]⁺; RT 4.32 min.

### f) rel-6-[(3aR*,7aS*)-2-oxo-octahydro-[1,3]oxazolo[5,4-c]pyridin-1-yl]-4-[(4-methoxyphenyl)methyl]-2H,3H,4H-pyrido[3,2-b][1,4]oxazin-3-one 1f

10% Pd/C (350 mg) was added to a solution of rel-benzyl (3aR*,7aS*)-1-{4-[(4-methoxyphenyl)methyl]-3-oxo-2H,3H,4H-pyrido[3,2-b][1,4]oxazin-6-yl}-2-oxo-octahydro-[1,3]oxazolo[5,4-c]pyridine-5-carboxylate 1e (3.35 g, 6.15 mmol) in DCM/MeOH (150 mL/200 mL) and the mixture was stirred under a H₂ atmosphere at room temperature for 73 h then heated at 50° C. for a further 24 h. The mixture was filtered and the filtrate was concentrated under reduced pressure to give the rel-6-[(3aR*,7aS*)-2-oxo-octahydro-[1,3]oxazolo[5,4-c]pyridin-1-yl]-4-[(4-methoxyphenyl)methyl]-2H,3H,4H-pyrido[3,2-b][1,4]oxazin-3-one 1f (2.40 g, 95%) as a pale yellow solid. TLC:R_{f}=0.3 (silica gel, DCM/MeOH=20:1, v/v). LC-MS (Method C) 411.2 [M+H]⁺; RT 2.41 min.

### g) rel-1-{2-[(3aR*,7aS*)-1-{4-[(4-methoxyphenyl)methyl]-3-oxo-2H,3H,4H-pyrido[3,2-b][1,4]oxazin-6-yl}-2-oxo-octahydro-[1,3]oxazolo[5,4-c]pyridin-5-yl]ethyl}-7-methoxy-1,2-dihydro-1,8-naphthyridin-2-one 1g

To a solution of 2-(7-methoxy-2-oxo-1,2-dihydro-1,8-naphthyridin-1-yl)acetaldehyde (prepared as described in WO2008009700) (200 mg, 0.92 mmol, 1.0 eq) and rel-6-[((3aR*,7aS*)-2-oxo-octahydro-[1,3]oxazolo[5,4-c]pyridin-1-yl]-4-[(4-methoxyphenyl)methyl]-2H,3H,4H-pyrido[3,2-b][1,4]oxazin-3-one 1f (378 mg, 0.92 mmol) in DCE (15 mL) at 0° C. was added NaBH(OAc)₃ (586 mg, 2.76 mmol) and HOAc (55 mg, 0.92 mmol) and the mixture was stirred at room temperature for 2 h. The mixture was diluted with H₂O, extracted with EtOAc (20 mL×3) and the combined extracts were washed with saturated aqueous NaHCO₃, H₂O, brine, dried over Na₂SO₄ and concentrated. The residue was purified by silica gel chromatography (DCM/MeOH=200:1 to 50:1) to give rel-1-{2-[((3aR*,7aS*)-1-{4-[(4-methoxyphenyl)methyl]-3-oxo-2H,3H,4H-pyrido[3,2-b][1,4]oxazin-6-yl}-2-oxo-octahydro-[1,3]oxazolo[5,4-c]pyridin-5-yl]ethyl}-7-methoxy-1,2-dihydro-1,8-naphthyridin-2-one 1g (140 mg, 25%) as a yellow solid. TLC:R_{f}=0.50 (silica gel, DCM/MeOH=20:1, v/v). LC-MS (Method C) 613.2 [M+H]⁺; RT 3.30 min.

### h) rel-1-{2-[(3aR*,7aS*)-2-oxo-1-{3-oxo-2H,3H,4H-pyrido[3,2-b][1,4]oxazin-6-yl}-octahydro-[1,3]oxazolo[5,4-c]pyridin-5-yl]ethyl}-7-methoxy-1,2-dihydro-1,8-naphthyridin-2-one 1

To a solution of rel-1-{2-[((3aR*,7aS*)-1-{4-[(4-methoxyphenyl)methyl]-3-oxo-2H,3H,4H-pyrido[3,2-b][1,4]oxazin-6-yl}-2-oxo-octahydro-[1,3]oxazolo[5,4-c]pyridin-5-yl]ethyl}-7-methoxy-1,2-dihydro-1,8-naphthyridin-2-one 1g (140 mg, 0.23 mmol) in TFA (5 mL) at 0° C. was added CF₃SO₃H (0.3 mL) and the mixture was stirred at room temperature for 0.5 h. The mixture was adjusted to pH 7-8 with a 2 M NaOH solution and then extracted with EtOAc (20 mL×3). The combined extracts were washed with saturated aqueous NaHCO₃, H₂O, brine, dried over Na₂SO₄, filtered and concentrated under reduced pressure. The residue was purified by preparative TLC (DCM/MeOH=20/1, v/v) to give rel-1-{2-[(3aR*,7aS*)-2-oxo-1-{3-oxo-2H,3H,4H-pyrido[3,2-b][1,4]oxazin-6-yl}-octahydro-[1,3]oxazolo[5,4-c]pyridin-5-yl]ethyl}-7-methoxy-1,2-dihydro-1,8-naphthyridin-2-one 1 (40 mg, 35%) as a white solid. TLC:R_{f}=0.40 (silica gel, DCM/MeOH=20:1, v/v). LC-MS (Method C) 493.2 [M+H]⁺; RT 2.43 min. NMR (Method E) (CDCl₃): δ ppm 7.85 (br s, 1H), 7.72 (m, 2H), 7.57 (d, J=9.2 Hz, 1H), 7.28 (d, J=8.8 Hz, 1H), 6.62 (d, J=8.4 Hz, 1H), 6.57 (d, J=9.6 Hz, 1H), 4.66 (m, 2H), 4.63 (s, 2H), 4.55 (m, 2H), 4.03 (s, 3H), 3.30 (d, J=13.6 Hz, 1H), 2.93 (m, 1H), 2.84 (m, 2H), 2.78 (d, J=13.2 Hz, 1H), 2.34 (m, 2H), 1.81 (m, 1H).

### Example 2:—rel-1-{3-[(3aR*,7aS*)-2-oxo-1-{3-oxo-2H,3H,4H-pyrido[3,2-b][1,4]oxazin-6-yl}-octahydro-[1,3]oxazolo[5,4-c]pyridin-5-yl]propyl}-1,2-dihydroquinolin-2-one

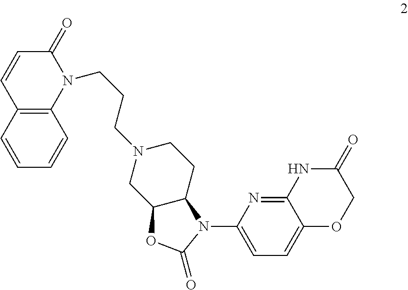 [SMILES: O=C1COc2ccc(N3C(=O)O[C@H]4CN(CCCn5c(=O)ccc6ccccc65)CC[C@H]43)nc2N1]

### a) rel-1-{3-[(3aR*,7aS*)-1-{4-[(4-methoxyphenyl)methyl]-3-oxo-2H,3H,4H-pyrido[3,2-b][1,4]oxazin-6-yl}-2-oxo-octahydro-[1,3]oxazolo[5,4-c]pyridin-5-yl]propyl}-1,2-dihydroquinolin-2-one 2a

To a solution of 3-(2-oxo-1,2-dihydroquinolin-1-yl)propanal (100 mg, 0.50 mmol, 1.0 eq) and rel-6-[(3aR*,7aS*)-2-oxo-octahydro-[1,3]oxazolo[5,4-c]pyridin-1-yl]-4-[(4-methoxyphenyl)methyl]-2H,3H,4H-pyrido[3,2-b][1,4]oxazin-3-one 1f (226 mg, 0.55 mmol) in DCE (10 mL) was added NaBH(OAc)₃ (318 mg, 1.50 mmol) and AcOH (30 mg, 0.50 mmol) dropwise and the mixture was stirred at room temperature for 17 h. H₂O was added and the mixture was adjusted to pH 8 with 1 M NaOH then extracted with EtOAc (100 mL). The organic layer was washed with brine, dried (Na₂SO₄), filtered and concentrated under reduced pressure. The residue was purified by silica gel chromatography (2% MeOH in DCM) to give rel-1-{3-[(3aR*,7aS*)-1-{4-[(4-methoxyphenyl)methyl]-3-oxo-2H,3H,4H-pyrido[3,2-b][1,4]oxazin-6-yl}-2-oxo-octahydro-[1,3]oxazolo[5,4-c]pyridin-5-yl]propyl}-1,2-dihydroquinolin-2-one 2a (169 mg, 57%) as a solid. TLC:R_{f}=0.4 (silica gel, DCM/MeOH=20:1, v/v). LC-MS (Method C) 596.3 [M+H]⁺; RT 2.97 min.

### b) rel-1-{3-[(3aR*,7aS*)-2-oxo-1-{3-oxo-2H,3H,4H-pyrido[3,2-b][1,4]oxazin-6-yl}-octahydro-[1,3]oxazolo[5,4-c]pyridin-5-yl]propyl}-1,2-dihydroquinolin-2-one 2

To a solution of rel-1-{3-[(3aR*,7aS*)-1-{4-[(4-methoxyphenyl)methyl]-3-oxo-2H,3H,4H-pyrido[3,2-b][1,4]oxazin-6-yl}-2-oxo-octahydro-[1,3]oxazolo[5,4-c]pyridin-5-yl]propyl}-1,2-dihydroquinolin-2-one 2a (147 mg, 0.25 mmol) in TFA (10 mL) at 0° C. under N₂ was added CF₃SO₃H (0.2 mL) dropwise and the mixture was heated at reflux for 2 h. After cooling to RT, H₂O was added and the mixture was adjusted to pH 10 with 1 M NaOH then extracted with EtOAc (200 mL). The organic layer was washed with brine, dried (Na₂SO₄), filtered and concentrated under reduced pressure. The residue was purified by silica gel chromatography (1% to 2% MeOH in DCM) followed by preparative HPLC to give rel-1-{3-[(3aR*,7aS*)-2-oxo-1-{3-oxo-2H,3H,4H-pyrido[3,2-b][1,4]oxazin-6-yl}-octahydro-[1,3]oxazolo[5,4-c]pyridin-5-yl]propyl}-1,2-dihydroquinolin-2-one 2 (10 mg) as a yellow solid. TLC:R_{f}=0.4 (silica gel, DCM/MeOH=20:1, v/v). LC-MS (Method C) 476.2 [M+H]⁺; RT 2.42 min. NMR (Method E) (CDCl₃): δ ppm 7.80 (br s, 1H), 7.78 (d, J=8.8 Hz, 1H), 7.68-7.55 (m, 4H), 7.32-7.21 (m, 2H), 6.69 (d, J=9.6 Hz, 1H), 4.64 (s, 2H), 4.59 (m, 2H), 4.45 (m, 1H), 4.32 (m, 1H), 3.32 (d, J=12.8 Hz, 1H), 2.80 (m, 1H), 2.60-2.42 (m, 4H), 2.14 (m, 1H), 2.03-1.75 (m, 3H).

### Example 3:—rel-(3aR*,7aS*)-5-(2-{2-methoxy-7-oxo-7H,8H-pyrido[2,3-d]pyrimidin-8-yl}ethyl)-1-{3-oxo-2H,3H,4H-pyrido[3,2-b][1,4]oxazin-6-yl}-octahydro-[1,3]oxazolo[5,4-c]pyridin-2-one

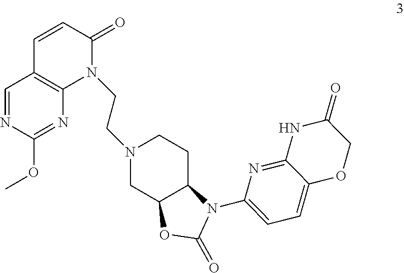 [SMILES: COc1ncc2ccc(=O)n(CCN3CC[C@@H]4[C@H](C3)OC(=O)N4c3ccc4c(n3)NC(=O)CO4)c2n1]

### a) 8-(2,2-diethoxyethyl)-2-methoxy-7H,8H-pyrido[2,3-d]pyrimidin-7-one 3a

K₂CO₃ (143 mg, 1.04 mmol) was added to a suspension of 2-methoxy-7H,8H-pyrido[2,3-d]pyrimidin-7-one (prepared as described in J. Med. Chem., 2011, 54, 7834) (100 mg, 0.56 mmol) in DMF (2 mL) and stirred at room temperature for 1 h. 2-bromo-1,1-diethoxyethane (0.09 mL, 0.62 mmol) was added and the mixture heated at 100° C. for 4 h. The reaction mixture was allowed to cool to room temperature then diluted with H₂O and extracted with DCM (3×20 mL). The combined organic extracts were washed with saturated brine (3 mL), dried (MgSO₄) and concentrated under reduced pressure to afford a brown residue containing 8-(2,2-diethoxyethyl)-2-methoxy-7H,8H-pyrido[2,3-d]pyrimidin-7-one 3a (91 mg, 55%) which was used without further purification.

### (b) 2-(2-methoxy-7-oxo-7H,8H-pyrido[2,3-d]pyrimidin-8-yl)acetaldehyde 3b

A solution of TFA (6 mL, 1.02 mmol) in H₂O (1.5 mL) was added to 8-(2,2-diethoxyethyl)-2-methoxy-7H,8H-pyrido[2,3-d]pyrimidin-7-one 3a (300 mg, 1.02 mmol) and the mixture stirred at room temperature for 3 h. Sat. aq. NaHCO₃ was added to the adjusting the mixture to pH 8 then extracted with DCM (20 mL) and IPA/DCM (1:3, 4×20 mL). The combined organic extracts were washed with brine, dried (MgSO₄) and concentrated under reduced pressure. The resulting residue was purified via silica gel chromatography using EtOAc to afford 2-(2-methoxy-7-oxo-7H,8H-pyrido[2,3-d]pyrimidin-8-yl)acetaldehyde 2b (173 mg, 77%) as a white solid. LC-MS (Method A) 220.4 [M+H]⁺; RT 1.39 min.

### c) rel-(3aR*,7aS*)-1-{3-oxo-2H,3H,4H-pyrido[3,2-b][1,4]oxazin-6-yl}-octahydro-[1,3]oxazolo[5,4-c]pyridin-2-one 3c

TFA (4.56 mL, 59.5 mmol) and CF₃COOH (1.05 mL, 11.9 mmol) were added to a solution of rel-benzyl (3aR*,7aS*)-1-{4-[(4-methoxyphenyl)methyl]-3-oxo-2H,3H,4H-pyrido[3,2-b][1,4]oxazin-6-yl}-2-oxo-octahydro-[1,3]oxazolo[5,4-c]pyridine-5-carboxylate 1e (0.93 g, 1.7 mmol) in DCM (20 mL) and the reaction mixture stirred at room temperature for 30 min. MeOH (10 mL) was added and the pH adjusted to 9 using sat. aq. Na₂CO₃. DCM (20 mL) was added and the mixture passed through an Isolute phase separator. The organic filtrate was concentrated under reduced pressure to afford rel-(3aR*,7aS*)-1-{3-oxo-2H,3H,4H-pyrido[3,2-b][1,4]oxazin-6-yl}-octahydro-[1,3]oxazolo[5,4-c]pyridin-2-one 3c (456 mg, 92.4%) which was used without further purification. LC-MS (Method A) 291.1 [M+H]⁺; RT 1.37 min.

### d) rel-(3aR*,7aS*)-5-(2-{2-methoxy-7-oxo-7H,8H-pyrido[2,3-d]pyrimidin-8-yl}ethyl)-1-{3-oxo-2H,3H,4H-pyrido[3,2-b][1,4]oxazin-6-yl}-octahydro-[1,3]oxazolo[5,4-c]pyridin-2-one 3

A solution of rel-(3aR*,7aS*)-1-{3-oxo-2H,3H,4H-pyrido[3,2-b][1,4]oxazin-6-yl}-octahydro-[1,3]oxazolo[5,4-c]pyridin-2-one 3c (97 mg, 0.33 mmol) and 2-(2-methoxy-7-oxo-7H,8H-pyrido[2,3-d]pyrimidin-8-yl)acetaldehyde 3b (37 mg, 0.17 mmol) in DCM (6 mL) and MeOH (2 mL) was stirred at room temperature for 2 h. NaBH(OAc)₃ (142 mg, 0.67 mmol) was added and the reaction mixture stirred for a further 1 h. The reaction was basified to pH 8 using sat. aq. NaHCO₃, diluted with DCM (20 mL) and passed through an Isolute phase separator. The organic filtrate was concentrated under reduced pressure and the residue purified via silica gel chromatography using 0-15% MeOH in DCM to give rel-(3aR*,7aS*)-5-(2-{2-methoxy-7-oxo-7H,8H-pyrido[2,3-d]pyrimidin-8-yl}ethyl)-1-{3-oxo-2H,3H,4H-pyrido[3,2-b][1,4]oxazin-6-yl}-octahydro-[1,3]oxazolo[5,4-c]pyridin-2-one 3 (17.6 mg, 11%). LCMS (Method B) 494.3 [M+H]⁺; RT 4.96 min. ¹H NMR (Method C) (DMSO-d6): δ ppm 11.24 (s, 1H), 8.96 (s, 1H), 7.99 (d, J=9.4 Hz, 1H), 7.50 (d, J=8.6 Hz, 1H), 7.46 (d, J=8.6 Hz, 1H), 6.61 (d, J=9.4 Hz, 1H), 4.72-4.66 (m, 1H), 4.67 (d, J=1.4 Hz, 2H), 4.58-4.51 (m, 1H), 4.50-4.40 (m, 2H), 4.06 (s, 3H), 3.23 (dt, J=13.3, 2.3 Hz, 1H), 2.93-2.86 (m, 1H), 2.78-2.65 (m, 3H), 2.49-2.39 (m, 1H), 2.21 (td, J=11.3, 3.0 Hz, 1H), 1.57 (dt, J=11.6, 2.6 Hz, 1H).

### Example 4:—rel-1-{2-[(3aR*,7aS*)-2-oxo-1-{3-oxo-2H,3H,4H-pyrido[3,2-b][1,4]oxazin-6-yl}-octahydro-[1,3]oxazolo[5,4-c]pyridin-5-yl]ethyl}-2-oxo-1,2-dihydroquinoline-7-carbonitrile

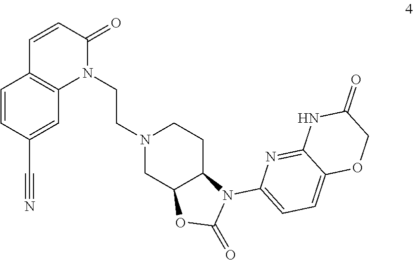 [SMILES: N#Cc1ccc2ccc(=O)n(CCN3CC[C@@H]4[C@H](C3)OC(=O)N4c3ccc4c(n3)NC(=O)CO4)c2c1]

A solution of rel-(3aR*,7aS*)-1-{3-oxo-2H,3H,4H-pyrido[3,2-b][1,4]oxazin-6-yl}-octahydro-[1,3]oxazolo[5,4-c]pyridin-2-one 3c (80 mg, 0.28 mmol) and 2-oxo-1-(2-oxoethyl)-1,2-dihydroquinoline-7-carbonitrile (49 mg, 0.23 mmol) (prepared according to J. Med. Chem, 2012, 55, 6916) (29 mg, 0.14 mmol) in DCM (4 mL) and MeOH (2 mL) was stirred over molecular sieves (3 Å) at room temperature for 2 h. NaBH(OAc)₃ (116 mg, 0.55 mmol) was added and stirring continued for 1 h. The reaction mixture was adjusted to pH 8 using NaHCO₃, diluted with DCM (20 mL), passed through an Isolute phase separator and the organic filtrate concentrated under reduced pressure. The residue was purified via silica gel chromatography using 0-20% MeOH in DCM to give rel-1-{2-[(3aR*,7aS*)-2-oxo-1-{3-oxo-2H,3H,4H-pyrido[3,2-b][1,4]oxazin-6-yl}-octahydro-[1,3]oxazolo[5,4-c]pyridin-5-yl]ethyl}-2-oxo-1,2-dihydroquinoline-7-carbonitrile 4 (7.2 mg, 5%). LCMS (Method B) 487.2 [M+H]⁺; RT 5.60 min. ¹H NMR (Method C) (DMSO-d6): δ ppm 11.19 (br. s, 1H), 8.13 (s, 1H), 8.01 (d, J=9.5 Hz, 1H), 7.92 (d, J=8.2 Hz, 1H), 7.66 (dd, J=8.2, 1.4 Hz, 1H), 7.48 (d, J=8.6 Hz, 1H), 7.42 (d, J=8.7 Hz, 1H), 6.79 (d, J=9.5 Hz, 1H), 4.67-4.63 (m, 1H), 4.63-4.60 (m, 2H), 4.53-4.41 (m, 2H), 4.41-4.32 (m, 1H), 3.26-3.15 (m, 1H), 2.93-2.87 (m, 1H), 2.68-2.61 (m, 3H), 2.41 (m, 1H), 2.20-2.10 (m, 1H), 1.62-1.50 (m, 1H).

### Example 5:—rel-1-{2-[(3aR*,7aS*)-2-oxo-1-{3-oxo-2H,3H,4H-pyrido[3,2-b][1,4]oxazin-6-yl}-octahydro-[1,3]oxazolo[5,4-c]pyridin-5-yl]ethyl}-7-methoxy-1,2-dihydro-1,5-naphthyridin-2-one

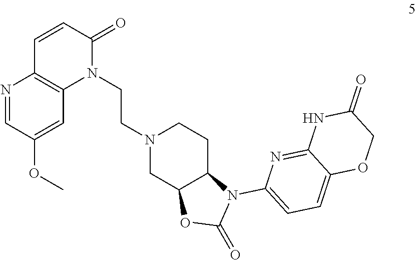 [SMILES: COc1cnc2ccc(=O)n(CCN3CC[C@@H]4[C@H](C3)OC(=O)N4c3ccc4c(n3)NC(=O)CO4)c2c1]

A solution of solution of rel-(3aR*,7aS*)-1-{3-oxo-2H,3H,4H-pyrido[3,2-b][1,4]oxazin-6-yl}-octahydro-[1,3]oxazolo[5,4-c]pyridin-2-one 3c (50 mg, 0.17 mmol) and 2-(7-methoxy-2-oxo-1,2-dihydro-1,5-naphthyridin-1-yl)acetaldehyde (prepared as described in WO 2010081874) (37.6 mg, 0.17 mmol) in DCM (10 mL) and MeOH (2 mL) was stirred at room temperature over molecular sieves (3 Å) for 2 h. NaBH(OAc)₃ (146.0 mg, 0.69 mmol) was added and the reaction stirred for 17 h. The pH was adjusted to 8 using sat. aq. NaHCO₃ and the mixture passed through an Isolute phase separator. The collected organic filtrate was concentrated under reduced pressure and the resulting residue purified via silica gel chromatography (0-20% MeOH in DCM) and then prep. HPLC (Method B, 0.1% NH₃ in MeCN/H₂O). Product fractions were concentrated under reduced pressure and co-evaporated from MeOH and Et₂O to afford rel-1-{2-[(3aR*,7aS*)-2-oxo-1-{3-oxo-2H,3H,4H-pyrido[3,2-b][1,4]oxazin-6-yl}-octahydro-[1,3]oxazolo[5,4-c]pyridin-5-yl]ethyl}-7-methoxy-1,2-dihydro-1,5-naphthyridin-2-one 5 (5 mg, 5% yield) as a white solid. LCMS (Method B) 493.2 [M+H]⁺; RT 5.16 min. ¹H NMR (Method C) (DMSO-d6): 5 ppm 11.20 (br. s, 1H), 8.31-8.26 (m, 1H), 7.88 (d, J=9.6 Hz, 1H), 7.49-7.44-7.41 (m, 2H), 7.42 (d, J=8.7 Hz 1H), 6.67 (d, J=9.6 Hz, 1H), 4.69-4.63 (m, 1H), 4.63-4.59 (m, 2H), 4.53-4.46 (m, 1H), 4.45-4.34 (m, 2H), 3.99 (s, 3H), 3.29-3.22 (m, 1H), 2.92-2.83 (m, 1H), 2.68-2.60 (m, 3H), 2.47-2.39 (m, 1H), 2.19-2.09 (m, 1H), 1.62-1.47 (m, 1H).

### Example 6:—rel-1-{3-[(3aR*,7aS*)-2-oxo-1-{3-oxo-2H,3H,4H-pyrido[3,2-b][1,4]oxazin-6-yl}-octahydro-[1,3]oxazolo[5,4-c]pyridin-5-yl]propyl}-2-oxo-1,2-dihydroquinoline-7-carbonitrile

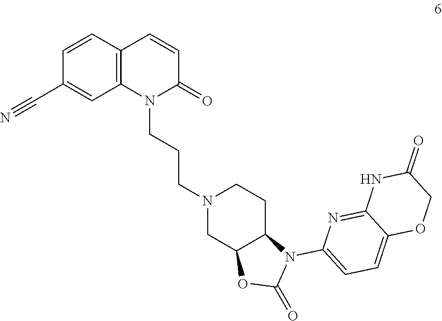 [SMILES: N#Cc1ccc2ccc(=O)n(CCCN3CC[C@@H]4[C@H](C3)OC(=O)N4c3ccc4c(n3)NC(=O)CO4)c2c1]

### a) 7-bromo-1-(3,3-dimethoxypropyl)-1,2-dihydroquinolin-2-one 6a

A solution of 7-bromo-1,2-dihydroquinolin-2-one (1.5 g, 6.68 mmol), 3-bromo-1,1-dimethoxypropane (0.96 mL, 7.03 mmol) and cesium carbonate (6.54 g, 20.03 mmol) in DMF (100 mL) was heated for 17 h at 100° C. After cooling, H₂O (250 mL) was added and the mixture extracted with EtOAc (3×100 mL). The combined extracts were dried (MgSO₄), filtered and concentrated under reduced pressure. The residue was purified via silica gel chromatography using 0-100% EtOAc in pet. ether to give 7-bromo-1-(3,3-dimethoxypropyl)-1,2-dihydroquinolin-2-one 6a (740 mg, 34%) as a colourless solid. LC-MS (Method A) 296.0 [M−OMe]⁺; RT 2.78 min.

### b) 1-(3,3-dimethoxypropyl)-2-oxo-1,2-dihydroquinoline-7-carbonitrile 6b

A solution of 7-bromo-1-(3,3-dimethoxypropyl)-1,2-dihydroquinolin-2-one 6a (740 mg, 2.27 mmol), tetrakis(triphenylphosphine)palladium(0) (131 mg, 0.11 mmol) and zinc cyanide (159 mg, 1.4 mmol) in DMF (3 mL) in a sealed microwave vial was irradiated at 150° C. for 90 min in a Biotage Initator. After cooling, H₂O (50 mL) was added and the mixture extracted with EtOAc (3×30 mL). The combined extracts were dried (MgSO₄), filtered and concentrated under reduced pressure to give a yellow solid. The residue was purified via silica gel chromatography using (0-100% EtOAc in Pet ether) to give 1-(3,3-dimethoxypropyl)-2-oxo-1,2-dihydroquinoline-7-carbonitrile 6b (340 mg, 51%) as a yellow solid. LC-MS (Method A) 241.1 [M−OMe]⁺; RT 2.32 min.

### c) 2-oxo-1-(3-oxopropyl)-1,2-dihydroquinoline-7-Carbonitrile 6c

A solution 1-(3,3-dimethoxypropyl)-2-oxo-1,2-dihydroquinoline-7-carbonitrile 6b in 2M hydrogen chloride in THF (5 mL) was stirred at room temperature for 2 h. H₂O (20 mL) was added and the mixture extracted with EtOAc (2×20 mL). The combined extracts were dried (MgSO₄), filtered and concentrated under reduced pressure to give 2-oxo-1-(3-oxopropyl)-1,2-dihydroquinoline-7-Carbonitrile 6c (210 mg, 82%) as an off white solid. LC-MS (Method A) 227.5 [M+H]⁺; RT 1.47 min.

### d) rel-1-{3-[(3aR*,7aS*)-2-oxo-1-{3-oxo-2H,3H,4H-pyrido[3,2-b][1,4]oxazin-6-yl}-octahydro-[1,3]oxazolo[5,4-c]pyridin-5-yl]propyl}-2-oxo-1,2-dihydroquinoline-7-carbonitrile 6

A solution of rel-(3aR*,7aS*)-1-{3-oxo-2H,3H,4H-pyrido[3,2-b][1,4]oxazin-6-yl}-octahydro-[1,3]oxazolo[5,4-c]pyridin-2-one 3c (77 mg, 0.27 mmol) and 2-oxo-1-(3-oxopropyl)-1,2-dihydroquinoline-7-Carbonitrile 6c (30 mg, 0.13 mmol) in DCM (4 mL) and MeOH (1 mL) was stirred over molecular sieves (3 Å) at room temperature for 2 h. NaBH(OAc)₃ (112 mg, 0.53 mmol) was added and stirred for a further 1 h. The reaction was adjusted to pH 8 using sat. aq. NaHCO₃ then passed through an Isolute phase separator. The organic filtrate was concentrated under reduced pressure and the residue purified via silica gel chromatography using 0-20% MeOH in DCM to give rel-1-{3-[(3aR*,7aS*)-2-oxo-1-{3-oxo-2H, 3H,4H-pyrido[3,2-b][1,4]oxazin-6-yl}-octahydro-[1,3]oxazolo[5,4-c]pyridin-5-yl]propyl}-2-oxo-1,2-dihydroquinoline-7-carbonitrile 6 (8.0 mg, 12%). LCMS (Method B) 501.1 [M+H]⁺; RT 5.78 min. ¹H NMR (Method C) (DMSO-d6): δ ppm 11.19 (br. s, 1H), 8.17-8.12 (m, 1H), 7.99 (d, J=9.6 Hz, 1H), 7.91 (d, J=8.1 Hz, 1H), 7.64 (dd, J=8.1, 1.3 Hz, 1H), 7.48 (d, J=8.6 Hz, 1H), 7.41 (d, J=8.6 Hz, 1H), 6.77 (d, J=9.6 Hz, 1H), 4.69-4.65 (m, 1H), 4.63-4.60 (m, 2H), 4.53-4.48 (m, 1H), 4.28 (t, J=7.5 Hz, 2H), 3.16-3.10 (m, 1H), 2.73-2.65 (m, 1H), 2.48-2.37 (m, 4H), 2.04-1.96 (m, 1H), 1.86-1.75 (m, 2H), 1.63-1.51 (m, 1H).

### Example 7:—rel-1-{3-[(3aR*,7aS*)-2-oxo-1-{3-oxo-2H,3H,4H-pyrido[3,2-b][1,4]oxazin-6-yl}-octahydro-[1,3]oxazolo[5,4-c]pyridin-5-yl]propyl}-7-methoxy-1,2-dihydro-1,8-naphthyridin-2-one

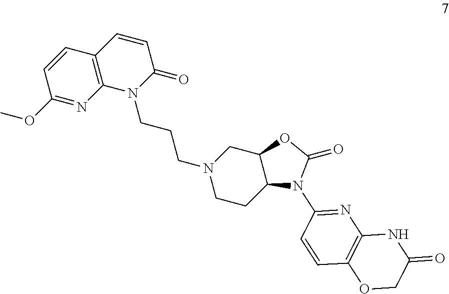 [SMILES: COc1ccc2ccc(=O)n(CCCN3CC[C@H]4[C@@H](C3)OC(=O)N4c3ccc4c(n3)NC(=O)CO4)c2n1]

### a) 1-(3,3-dimethoxypropyl)-7-methoxy-1,2-dihydro-1,8-naphthyridin-2-one 7a

7-methoxy-1,2-dihydro-1,8-naphthyridin-2-one (0.9 g, 5.11 mmol), CsCO₃ (1.6 g, 4.91 mmol) and 3-bromo-1,1-dimethoxypropane (0.91 mL, 6.64 mmol) were suspended in DMF (50 mL) and heated to 90° C. for 1 h. H₂O (100 mL) was added and the mixture extracted with Et₂O (2×100 mL). Organic extracts were combined, dried (MgSO₄), filtered and concentrated under reduced pressure to afford an orange gum. Purification via column chromatography eluting with 50-100% Et₂O in heptane afforded 1-(3,3-dimethoxypropyl)-7-methoxy-1,2-dihydro-1,8-naphthyridin-2-one 7a (0.87 g, 2.97 mmol, 58%) as a yellow solid. LC-MS (Method A) 301.2 [M+Na]⁺; RT 2.45 min.

### b) 3-(7-methoxy-2-oxo-1,2-dihydro-1,8-naphthyridin-1-yl)propanal 7b

HCl (3M aqueous, 10 mL, 2.3 mmol) was added to a solution of 1-(3,3-dimethoxypropyl)-7-methoxy-1,2-dihydro-1,8-naphthyridin-2-one 7a (0.64 g, 2.3 mmol) in THF (15 mL) and stirred at 60° C. for 27 h. The mixture was allowed to cool to room temperature and then quenched with sat. aqueous Na₂CO₃ (100 mL). The reaction mixture was extracted with DCM (2×100 mL) and the combined organics dried (MgSO₄), filtered and concentrated under reduced pressure to afford 3-(7-methoxy-2-oxo-1,2-dihydro-1,8-naphthyridin-1-yl)propanal 7b (320 mg, 1.24 mmol, 54% yield) as a yellow gum which solidified. ¹H NMR (Method C)¹H-NMR (CDCl₃) δ: 9.78 (s 1H), 8.05 (d, J=8.3 Hz, 1H), 7.88 (d, J=9.5 Hz, 1H), 6.72 (d, J=8.3 Hz, 1H), 6.50 (d, J=9.5 Hz, 1H), 4.65 (t, J=7.3 Hz, 2H), 3.95 (s, 3H), 2.82 (t, J=7.3 Hz, 2H).

### c) rel-1-{3-[(3aR*,7aS*)-2-oxo-1-{3-oxo-2H,3H,4H-pyrido[3,2-b][1,4]oxazin-6-yl}-octahydro-[1,3]oxazolo[5,4-c]pyridin-5-yl]propyl}-7-methoxy-1,2-dihydro-1,8-naphthyridin-2-one 7

A solution of rel-(3aR*,7aS*)-1-{3-oxo-2H,3H,4H-pyrido[3,2-b][1,4]oxazin-6-yl}-octahydro-[1,3]oxazolo[5,4-c]pyridin-2-one 3c (125 mg, 0.43 mmol) and 3-(7-methoxy-2-oxo-1,2-dihydro-1,8-naphthyridin-1-yl)propanal 7b (50 mg, 0.22 mmol) in DCM (8 mL) and MeOH (2 mL) was stirred at room temperature for 2 h. NaBH(OAc)₃ (136 mg, 0.65 mmol) was added and stirred for a further 1 h. The reaction was adjusted to pH 8 using sat. aq. NaHCO₃, diluted with DCM (30 mL) and the resulting layers separated through an Isolute phase separator. The organic filtrate was concentrated under reduced pressure and purified via silica gel chromatography using 0-15% MeOH in DCM to give rel-1-{3-[(3aR*,7aS*)-2-oxo-1-{3-oxo-2H, 3H,4H-pyrido[3,2-b][1,4]oxazin-6-yl}-octahydro-[1,3]oxazolo[5,4-c]pyridin-5-yl]propyl}-7-methoxy-1,2-dihydro-1,8-naphthyridin-2-one 7 (8.1 mg, 7%). LCMS (Method B) 507.1 [M+H]⁺; RT 5.12 min. ¹H NMR (Method C) (DMSO-d6): δ ppm 11.19 (br. s, 1H), 8.04 (d, J=8.5 Hz, 1H), 7.85 (d, J=9.3 Hz, 1H), 7.48 (d, J=8.7 Hz, 1H), 7.41 (d, J=8.6 Hz, 1H), 6.72 (d, J=8.5 Hz, 1H), 6.49 (d, J=9.3 Hz, 1H), 4.67-4.62 (m, 1H), 4.62 (d, J=1.3 Hz, 2H), 4.53-4.43 (m, 1H), 4.39 (t, J=7.4 Hz, 2H), 3.98 (s, 3H), 3.16-3.09 (m, 1H), 2.75-2.65 (m, 1H), 2.49-2.35 (m, 4H), 2.03-1.92 (m, 1H), 1.90-1.82 (m, 2H), 1.60-1.43 (m, 1H).

### Example 8:—rel-(3aR*,7aR*)-5-(2-{2-methoxy-7-oxo-7H,8H-pyrido[2,3-d]pyrimidin-8-yl}ethyl)-1-{3-oxo-2H,3H,4H-pyrido[3,2-b][1,4]oxazin-6-yl}-octahydro-[1,3]oxazolo[5,4-c]pyridin-2-one

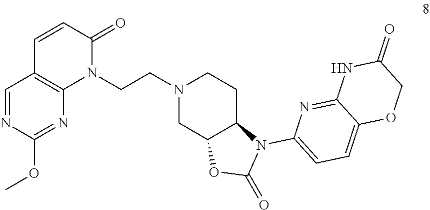 [SMILES: COc1ncc2ccc(=O)n(CCN3CC[C@@H]4[C@@H](C3)OC(=O)N4c3ccc4c(n3)NC(=O)CO4)c2n1]

### a) rel-benzyl (3R*,4R*)-4-amino-3-hydroxypiperidine-1-carboxylate 8a

TFA (60 mL) was added to a solution of rel-benzyl (3R*,4R*)-4-{[(tert-butoxy)carbonyl]amino}-3-hydroxypiperidine-1-carboxylate (23.1 g, 66.0 mmol)(prepared as described in WO 2010016005 A1) in DCM (120 mL) and stirred at room temperature for 1 h. The reaction mixture was concentrated under reduced pressure and the resulting residue diluted with EtOAc (300 mL) and extracted with aq. HCl (0.5 N, 400 mL). The aqueous phase was adjusted to pH 12 and extracted with DCM (3×300 mL). The organic extracts were combined, washed with sat. brine (400 mL), dried (MgSO₄) and concentrated under reduced pressure to afford rel-benzyl (3R*,4R*)-4-amino-3-hydroxypiperidine-1-carboxylate 8a (10.1 g, 61%) as a light red oil. ¹H NMR (Method E) (CD₃OD): δ ppm 7.35-7.28 (m, 5H), 5.10 (s, 2H), 4.77-4.15 (m, 1H), 4.09-4.05 (m, 1H), 3.18-3.12 (m, 1H), 2.84 (m, 1H), 2.61-2.54 (m, 2H), 1.84 (m, 1H), 1.32 (m, 1H).

### b) rel-benzyl (3aR*,7aR*)-2-oxo-octahydro-[1,3]oxazolo[5,4-c]pyridine-5-carboxylate 8b

Triphosgene (474.3 mg, 1.6 mmol) was added to a stirred solution of rel-benzyl (3R*,4R*)-4-amino-3-hydroxypiperidine-1-carboxylate 8a (1.0 g, 4.0 mmol) and triethylamine (1.67 mL, 11.99 mmol) in THF (40 mL) and heated at 60° C. for 17 h under nitrogen. The reaction mixture was allowed to cool to room temperature, concentrated under reduced pressure and the resulting residue diluted with water (30 mL). The mixture was extracted with EtOAc (2×50 mL), and the extracts washed with sat. brine dried (MgSO₄), and concentrated under reduced pressure to afford rel-benzyl (3aR*,7aR*)-2-oxo-octahydro-[1,3]oxazolo[5,4-c]pyridine-5-carboxylate 8b (1.07g, 97%) as a brown oil which was used directly without purification. LCMS (Method A) 277.1 [M+H]⁺; RT 2.32 min.

### c) rel-benzyl (3aR*,7aR*)-1-{4-[(4-methoxyphenyl)methyl]-3-oxo-2H,3H,4H-pyrido[3,2-b][1,4]oxazin-6-yl}-2-oxo-octahydro-[1,3]oxazolo[5,4-c]pyridine-5-carboxylate 8c

To a stirred solution of rel-benzyl (3aR*,7aR*)-2-oxo-octahydro-[1,3]oxazolo[5,4-c]pyridine-5-carboxylate 8b (1.07 g, 3.88 mmol) and 6-bromo-4-[(4-methoxyphenyl)methyl]-2H,3H,4H-pyrido[3,2-b][1,4]oxazin-3-one (prepared as described in WO2014108832) (1.36 g, 3.88 mmol) in 1,4-Dioxane (40 mL) was added K₃PO₄ (1.65 g, 7.76 mmol) and XPhos Pd G2 (152.6 mg, 0.19 mmol) and the reaction mixture heated at 100° C. for 2.5 h. The reaction mixture was allowed to cool to room temperature, diluted with H₂O (30 mL) and DCM (50 mL) and the organic layer separated through an SPE phase separator. The organic filtrate was concentrated under reduced pressure and the resulting brown oil purified via silica gel chromatography using 0-100% EtOAc in pet. ether. Product containing fractions eluted at approximately 80% EtOAc in pet.ether and were concentrated under reduced pressure to afford rel-benzyl (3aR*,7aR*)-1-{4-[(4-methoxyphenyl)methyl]-3-oxo-2H,3H,4H-pyrido[3,2-b][1,4]oxazin-6-yl}-2-oxo-octahydro-[1,3]oxazolo[5,4-c]pyridine-5-carboxylate 8c (1.38 g, 65%—contains some unreacted 8b). LCMS (Method A): RT 3.95 min

### d) rel-(3aR*,7aR*)-1-{3-oxo-2H,3H,4H-pyrido[3,2-b][1,4]oxazin-6-yl}-octahydro-[1,3]oxazolo[5,4-c]pyridin-2-one 8d

To a stirred solution of rel-benzyl (3aR*,7aR*)-1-{4-[(4-methoxyphenyl)methyl]-3-oxo-2H,3H,4H-pyrido[3,2-b][1,4]oxazin-6-yl}-2-oxo-octahydro-[1,3]oxazolo[5,4-c]pyridine-5-carboxylate 8c (1.38 g, 2.53 mmol) in DCM (30 mL) was added TFA (2 mL, 26.1 mmol) and CF3COOH (0.5 mL, 5.65 mmol) and the reaction stirred at room temperature for 17 h. The reaction was quenched with MeOH (5 mL) and adjusted to pH 9 with sat. Na₂CO₃. DCM (10 mL) was added and the mixture passed through an SPE phase separator and the filtrate concentrated under reduced pressure to afford rel-(3aR*,7aR*)-1-{3-oxo-2H,3H,4H-pyrido[3,2-b][1,4]oxazin-6-yl}-octahydro-[1,3]oxazolo[5,4-c]pyridin-2-one 8d (873 mg, 118%) which was used directly without purification. LCMS (Method A) 291.2 [M+H]⁺; RT 1.46 min.

### e) rel-(3aR*,7aR*)-5-(2-{2-methoxy-7-oxo-7H,8H-pyrido[2,3-d]pyrimidin-8-yl}ethyl)-1-{3-oxo-2H,3H,4H-pyrido[3,2-b][1,4]oxazin-6-yl}-octahydro-[1,3]oxazolo[5,4-c]pyridin-2-one 8

A solution of rel-(3aR*,7aR*)-1-{3-oxo-2H,3H,4H-pyrido[3,2-b][1,4]oxazin-6-yl}-octahydro-[1,3]oxazolo[5,4-c]pyridin-2-one 8d (92.7 mg, 0.32 mmol) and 2-(2-methoxy-7-oxo-7H,8H-pyrido[2,3-d]pyrimidin-8-yl)acetaldehyde 3b (35 mg, 0.16 mmol) in DCM (8 mL) was stirred at room temperature for 2 h. NaBH(OAc)₃ (101.5 mg, 0.48 mmol) was added and the mixture stirred for 17 h. Additional NaBH(OAc)₃ (101.5 mg, 0.48 mmol) was added and the reaction mixture stirred at room temperature for 2 h. Further 2-(2-methoxy-7-oxo-7H,8H-pyrido[2,3-d]pyrimidin-8-yl)acetaldehyde 3b (20 mg, 0.09 mmol) was added and the reaction mixture stirred at room temperature for 17 h. The mixture was neutralised with sat. aq. NaHCO₃ and the reaction mixture diluted with DCM (30 mL). The resulting organic layer was separated through a SPE phase separator. The filtrate was concentrated under reduced pressure and the resulting crude residue purified via silica gel chromatography using 0-20% MeOH in DCM followed by further purification by prep. HPLC (Method B, 0.1% NH3 in MeCN/H₂O). Clean fractions were concentrated under reduced pressure and co-evaporated from MeOH and Et₂O to afford rel-(3aR*,7aR*)-5-(2-{2-methoxy-7-oxo-7H,8H-pyrido[2,3-d]pyrimidin-8-yl}ethyl)-1-{3-oxo-2H,3H,4H-pyrido[3,2-b][1,4]oxazin-6-yl}-octahydro-[1,3]oxazolo[5,4-c]pyridin-2-one 8 (3.2 mg, 2%) as a white solid. LC-MS (Method B) 494.2 [M+H]⁺; RT 5.09 min. ¹H NMR (Method C) (DMSO-d6): δ ppm 11.23 (s, 1H), 8.93 (s, 1H), 7.96 (d, J=9.5 Hz, 1H), 7.42 (d, J=8.6 Hz, 1H), 7.19 (d, J=8.6 Hz, 1H), 6.58 (d, J=9.5 Hz, 1H), 4.61 (s, 2H), 4.49-4.38 (m, 2H), 4.04 (s, 3H), 4.02-3.95 (m, 1H), 3.81-3.71 (m, 1H), 3.50-3.45 (m, 1H), 3.12-3.06 (m, 1H), 2.89-2.75 (m, 3H), 2.54-2.52 (m, 2H) 1.55-1.44 (m, 1H).

### Example 9:—rel-1-{3-[(3aR*,7aR*)-2-oxo-{3-oxo-2H,3H,4H-pyrido[3,2-b][1,4]oxazin-6-yl}-octahydro-[1,3]oxazolo[5,4-c]pyridin-5-yl]propyl}-7-methoxy-1,2-dihydro-1,8-naphthyridin-2-one

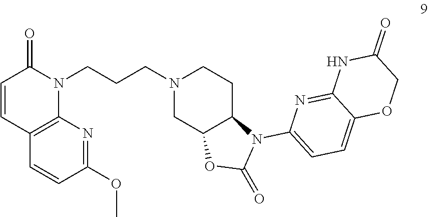 [SMILES: COc1ccc2ccc(=O)n(CCCN3CC[C@@H]4[C@@H](C3)OC(=O)N4c3ccc4c(n3)NC(=O)CO4)c2n1]

A solution of rel-(3aR*,7aR*)-1-{3-oxo-2H,3H,4H-pyrido[3,2-b][1,4]oxazin-6-yl}-octahydro-[1,3]oxazolo[5,4-c]pyridin-2-one 8d (94 mg, 0.32 mmol) and 3-(7-methoxy-2-oxo-1,2-dihydro-1,8-naphthyridin-1-yl)propanal 7b (50 mg, 0.22 mmol) in DCM (6 mL) was stirred at room temperature for 2 h. NaBH(OAc)₃ (136 mg, 0.65 mmol) was added and stirred for a further 17 h at room temperature. The reaction mixture was neutralised using sat. aq. NaHCO₃, diluted with DCM (20 mL) and the organic layer separated through a SPE phase separator. The organic filtrate was concentrated under reduced pressure and the resulting residue purified via silica gel chromatography using 0-20% MeOH in DCM to afford rel-1-{3-[(3aR*,7aR*)-2-oxo-1-{3-oxo-2H,3H,4H-pyrido[3,2-b][1,4]oxazin-6-yl}-octahydro-[1,3]oxazolo[5,4-c]pyridin-5-yl]propyl}-7-methoxy-1,2-dihydro-1,8-naphthyridin-2-one 9 (8.1 mg, 7%) as a white solid. LCMS (Method B) 507.3 [M+H]⁺; RT 5.86 min. ¹H NMR (Method C) (DMSO-d6): δ ppm 11.22 (s, 1H), 8.05 (d, J=8.4 Hz, 1H), 7.86 (d, J=9.4 Hz, 1H), 7.42 (d, J=8.5 Hz, 1H), 7.22 (d, J=8.5 Hz, 1H), 6.72 (d, J=8.4 Hz, 1H), 6.50 (d, J=9.4 Hz, 1H), 4.62 (s, 2H), 4.47-4.33 (m, 2H), 4.07-3.94 (m, 1H), 3.99 (s, 3H), 3.78-3.68 (m, 1H), 3.38-3.29 (m, 1H), 3.02-2.94 (m, 1H), 2.83-2.75 (m, 1H), 2.70-2.56 (m, 2H), 2.36-2.27 (m, 1H), 2.23-2.13 (m, 1H), 1.95-1.84 (m, 2H), 1.54-1.44 (m, 1H).

### Example 10:—rel-1-[{3-(3aR*,7aR*)-2-oxo-1-{3-oxo-2H,3H,4H-pyrido[3,2-b][1,4]oxazin-6-yl}-octahydro-[1,3]oxazolo[5,4-c]pyridin-5-yl]propyl}-2-oxo-1,2-dihydroquinoline-7-carbonitrile

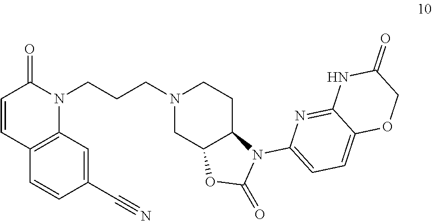 [SMILES: N#Cc1ccc2ccc(=O)n(CCCN3CC[C@@H]4[C@@H](C3)OC(=O)N4c3ccc4c(n3)NC(=O)CO4)c2c1]

A solution of rel-(3aR*,7aR*)-1-{3-oxo-2H,3H,4H-pyrido[3,2-b][1,4]oxazin-6-yl}-octahydro-[1,3]oxazolo[5,4-c]pyridin-2-one 8d (100.0 mg, 0.34 mmol) and 7-bromo-1-(3,3-dimethoxypropyl)-1,2-dihydroquinolin-2-one 6a (58 mg, 0.26 mmol) in DCM (6 mL) was stirred at room temperature for 2 h. NaBH(OAc)₃ (219 mg, 1.03 mmol) was added and the reaction stirred for a further 17 h. The reaction was neutralised using sat. aq. NaHCO₃, diluted with DCM (10 mL), and the resulting organic layer separated through a SPE phase separator. The organic filtrate was concentrated under reduced pressure and the resulting residue was purified via silica gel chromatography using 0-15% MeOH in DCM to afford rel-1-{3-[(3aR*,7aR*)-2-oxo-1-{3-oxo-2H,3H,4H-pyrido[3,2-b][1,4]oxazin-6-yl}-octahydro-[1,3]oxazolo[5,4-c]pyridin-5-yl]propyl}-2-oxo-1,2-dihydroquinoline-7-carbonitrile 10 (7.5 mg, 4%) as a white solid. LCMS (Method B) 501.2 [M+H]⁺; RT 5.74 min. ¹H NMR (Method C) (DMSO-d6): δ ppm 11.28 (s, 1H), 8.23 (d, J=1.4 Hz, 1H), 8.06 (d, J=9.5 Hz, 1H), 7.97 (d, J=8.0 Hz, 1H), 7.71 (dd, J=8.0, 1.3 Hz, 1H), 7.47 (d, J=8.6 Hz, 1H), 7.27 (d, J=8.6 Hz, 1H), 6.84 (d, J=9.5 Hz, 1H), 4.67 (s, 2H), 4.41-4.24 (m, 2H), 4.19-4.11 (m, 1H), 3.86-3.74 (m, 1H), 3.40-3.35 (m, 1H), 3.05-2.98 (m, 1H), 2.90-2.83 (m, 1H), 2.70-2.57 (m, 2H), 2.47-2.36 (m, 1H), 2.33-2.23 (m, 1H), 1.92-1.81 (m, 2H), 1.68-1.56 (m, 1H).

### Example 11—Antibacterial Susceptibility Testing

Minimum Inhibitory Concentrations (MICs) versus planktonic bacteria are determined by the broth microdilution procedure according to the guidelines of the Clinical and Laboratory Standards Institute (Clinical and Laboratory Standards Institute. Methods for Dilution Antimicrobial Susceptibility Tests for Bacteria That Grow Aerobically; Approved Standard-Ninth Edition. CLSI document M07-A10, 2015). The broth dilution method involves a two-fold serial dilution of compounds in 96-well microtitre plates, giving a typical final concentration range of 0.25-128 μg/mL and a maximum final concentration of 1% DMSO. The bacterial strains tested include the Gram-positive strains Staphylococcus aureus ATCC 29213 and Streptococcus pneumoniae ATCC 49619 and the Gram negative strains Acinetobacter baumannii NCTC 13420, Acinetobacter baumannii ATCC 19606, Enterobacter cloacae NCTC 13406, Escherichia coli ATCC 25922, Haemophilus influenzae ATCC 49247, Klebsiella pneumoniae ATCC 700603, Pseudomonas aeruginosa ATCC 27853 and P. aeruginosa NCTC 13437.

Strains are grown in cation-adjusted Müller-Hinton broth at 37° C. in an ambient atmosphere. The MIC is determined as the lowest concentration of compound that inhibits growth following a 16-20 h incubation period. The data reported correspond to the modes of three independent experiments and is reported in Table 1.

In Table 1, an MIC (in μg/mL) of less or equal to 1 is assigned the letter A; a MIC of from 1 to 10 is assigned the letter B; a MIC of from 10 to 100 is assigned the letter C; and a MIC of over 100 is assigned the letter D.

All compounds tested show activity against both Gram-negative and Gram-positive bacteria.

TABLE 1
MIC values of reference compound and test compounds 1-10
against Gram-negative and Gram-positive bacterial strains

| Strains | CIP | 1 | 2 | 3 | 4 | 5 | 6 | 7 | 8 | 9 | 10 |
|---|---|---|---|---|---|---|---|---|---|---|---|
| A. baumannii | C | A | A | B | A | A | A | A | B | A | A |
| NCTC 13420 | | | | | | | | | | | |
| A. baumannii | A | A | B | B | A | B | B | A | B | A | A |
| ATCC 19606 | | | | | | | | | | | |
| E. cloacae | A | B | C | C | B | C | B | B | C | B | B |
| NCTC 13406 | | | | | | | | | | | |
| E. coli | A | B | B | C | A | B | A | A | C | A | A |
| ATCC 25922 | | | | | | | | | | | |
| H. influenzae | A | B | B | B | A | A | B | A | B | A | A |
| ATCC 49247 | | | | | | | | | | | |
| K. pneumoniae | A | C | C | C | C | C | C | C | C | C | C |
| ATCC 700603 | | | | | | | | | | | |
| P. aeruginosa | A | C | C | C | B | C | C | C | C | B | B |
| ATCC 27853 | | | | | | | | | | | |
| P. aeruginosa | C | B | C | C | B | B | C | B | C | B | B |
| NCTC 13437 | | | | | | | | | | | |
| S. aureus | A | A | A | A | A | A | A | A | A | A | A |
| ATCC 29213 | | | | | | | | | | | |
| S. pneumoniae | A | A | A | A | A | A | A | A | A | A | A |
| ATCC 49619 | | | | | | | | | | | |

### Example 12—Human Cell Viability Assay

Compounds are assessed for potential non-specific cytotoxic effects against a human hepatic cell line (HepG2 ATCC HB-8065). HepG2 cells are seeded at 20,000 cells/well in 96-well microtitre plates in minimal essential medium (MEM) supplemented with a final concentration of 10% FBS and 1 mM sodium pyruvate. After 24 h compound dilutions are prepared in Dulbecco's minimum essential media (DMEM) supplemented with final concentrations of 0.001% FBS, 0.3% bovine albumin and 0.02% HEPES and added to cells. Compounds are tested in two-fold serial dilutions over a final concentration range of 1-128 μg/mL in a final DMSO concentration of 1% vol/vol. Chlorpromazine is used as a positive control. Cells are incubated with compound at 37° C. and 5% CO₂ for a further 24 h, after which time the CellTiter-Glo reagent (Promega) is added. Luminescence is measured on a BMG Omega plate reader. Data are analysed using GraphPad Prism software to determine the concentration of compound that inhibits cell viability by fifty percent (IC₅₀). The results are provided in Table 2.

In Table 2, an IC₅₀ (in μg/mL) of less than 1 is assigned the letter D; an IC₅₀ of from 1 to 10 is assigned the letter C; an IC₅₀ of from 10 to 100 is assigned the letter B; and an IC₅₀ of over 100 is assigned the letter A.

TABLE 2
IC₅₀ values against HepG2 cell line

| | IC₅₀ (μg/mL) |
|---|---|
| Compound | HepG2 |
| 1 | A |
| 2 | B |
| 3 | A |
| 4 | B |
| 6 | B |
| 7 | B |
| 9 | B |
| 10 | B |

Compounds 1-10 show low toxicities against HepG2 human hepatic cell line. In particular, compounds 1 and 3 showed no detectable toxicity against the tested human hepatic cell line. Therefore compounds 1 and 3 showed an excellent therapeutic benefit relative to their hepatic toxicity. Compounds 2 4, 6, 7, 9, and 10 also demonstrated an acceptable level of hepatic toxicity relative to therapeutic activity. This indicates that these compounds have the potential to have an excellent therapeutic benefit relative to their hepatic toxicity.

1. A compound of formula (I), or a pharmaceutically acceptable salt or N-oxide thereof: 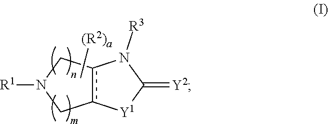
wherein [symbol] is a double bond or a single bond;
Y¹ is independently selected from the group consisting of O and S;
Y² is independently selected from the group consisting of O and S;
R¹ is independently selected from the group consisting of -L-Ar¹—Ar² and 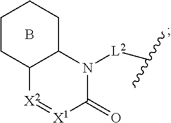
Ar¹ and Ar² are each independently a phenyl or monocyclic heteroaryl group;
-L¹- is —C₁-C₃-alkylene-;
X¹ is independently selected from the group consisting of N and CR⁴; and X² is independently selected from the group consisting of N and CR⁵; or
X¹ and X² together form a 5-membered heteroaryl ring;
-L²- is —C₂-C₃-alkylene-;
Ring B is independently selected from the group consisting of phenyl, monocyclic 6-membered heteroaryl and pyridinone, optionally substituted with a single —Y³—R⁶ group; Y³ is absent or is independently selected from the group consisting of NR⁷, O and S; where Ring B is a pyridinone ring, the nitrogen of the Ring B pyridinone may be attached to the proximal end of a —C₁-C₃-alkylene-group that is attached at its distal end to the group -L²-R² is independently at each occurrence selected from the group consisting of halo, nitro, cyano, NR⁸R⁹, NR⁸S(O)₂R⁸, NR⁸CONR⁸R⁸, NR⁸C(O)R⁸, NR⁸CO₂R⁸, OR, SR⁸, SOR⁸, SO₃R⁸, SO₂R⁸, SO₂NR⁸R⁸, CO₂R⁸, C(O)R⁸, CONR⁸R⁸, C₁-C₄-alkyl, C₂-C₄-alkenyl, C₂-C₄-alkynyl, C₁-C₄-haloalkyl and O—C₁-C₄-haloalkyl;
R³ is a bicyclic carbocyclic or a bicyclic heterocyclic ring system in which at least one of the two rings is aryl or heteroaryl;
or R³ is -L³-phenyl; wherein -L³- is selected from the group consisting of —CR⁸═CR⁸— and —C₄-cycloalkyl-;
R⁴ and R⁵ are each independently selected from the group consisting of H, halo, cyano, C₁-C₄-alkyl and O—C₁-C₄-alkyl;
R⁶ is independently selected from the group consisting of H, C₁-C₄-alkyl, C₂-C₄-alkenyl, C₂-C₄-alkynyl, C₁-C₄-haloalkyl, C₃-C₈-cycloalkyl, ₄₋₇-heterocycloalkyl, phenyl, monocyclic heteroaryl and C₁-C₃-alkylene-R^{6a}; wherein R^{6a} is independently selected from the group consisting of C₃-C₈-cycloalkyl, ₄₋₇-heterocycloalkyl, phenyl and monocyclic heteroaryl;
R⁷ is independently selected from the group consisting of H and C₁-C₄-alkyl;
or R⁶ and R⁷ together with the nitrogen to which they are attached form a 4- to 7-membered heterocycloalkyl ring;
R⁸ is independently at each occurrence selected from the group consisting of H and C₁-C₄-alkyl;
R⁹ is independently selected from the group consisting of H, C₁-C₄-alkyl, C₁-C₄-haloalkyl, S(O)₂—C₁-C₄-alkyl and C(O)—C₁-C₄-alkyl;
a is an integer from 0 to 4;
n and m are each an integer selected from the group consisting of 1 and 2; wherein the sum of n and m is 2 or 3;
wherein any of the aforementioned alkyl, alkylene, alkenyl, alkynyl, haloalkyl, cycloalkyl, carbocyclic, heterocyclic, heterocycloalkyl, aryl, phenyl and heteroaryl groups is optionally substituted, where chemically possible, by 1 to 5 substituents which are each independently at each occurrence selected from the group consisting of oxo, ═NR^{a}, ═NOR^{a}, halo, nitro, cyano, NR^{a}R^{a}, NR^{a}S(O)₂R^{a}, NR^{a}C(O)R^{a}, NR^{a}CONR^{a}R^{a}, NR^{a}CO₂R^{a}, OR^{a}, SR^{a}, SOR^{a}, SO₃R^{a}, SO₂R^{a}, SO₂NR^{a}R^{a}, —CO₂R^{a}, C(O)R^{a}, CONR^{a}R^{a}, CR^{a}R^{a}NR^{a}R^{a}, CR^{a}R^{a}OR^{a}, C₁-C₄-alkyl, C₂-C₄-alkenyl, C₂-C₄-alkynyl, C₁-C₄-haloalkyl and O—C₁-C₄-haloalkyl; wherein R^{a} is independently at each occurrence selected from the group consisting of H C₁-C₄ alkyl.

2. The compound of claim 1, wherein the compound is represented by formula (IX):

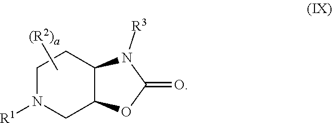

3. The compound of claim 1, wherein the compound is represented by formula (X):

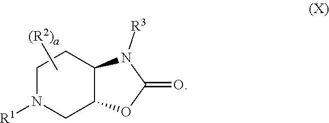

4. The compound of claim 1, wherein a is 0.

5. The compound of claim 1, wherein R¹ is: 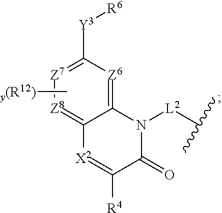
wherein y is an integer from 0 to 2; Z⁶, Z⁷ and Z⁸ are each independently carbon or nitrogen; provided that no more than 2 of Z⁶, Z⁷ and Z⁸ are nitrogen; and R¹² is independently selected from the group consisting of halo, nitro, cyano, NR^{a}R^{a}, NR^{a}S(O)₂R^{a}, NR^{a}C(O)R^{a}, NR^{a}CONR^{a}R^{a}, NR^{a}CO₂R^{a}, OR^{a}, SR^{a}, SOR^{a}, SO₃R^{a}, SO₂R^{a}, SO₂NR^{a}R^{a}, CO₂R^{a}, C(O)R^{a}, CONR^{a}R^{a}, CR^{a}R^{a}NR^{a}R^{a}, CR^{a}R^{a}OR^{a}, C₁-C₄-alkyl, C₂-C₄-alkenyl, C₂-C₄-alkynyl, C₁-C₄-haloalkyl and O—C₁-C₄-haloalkyl.

6. (canceled)

7. The compound of claim 5, wherein Y³ is O; and R⁶ is independently selected from the group consisting of C₁-C₄-alkyl, C₂-C₄-alkenyl, C₂-C₄-alkynyl, C₁-C₄-haloalkyl, C₃-C₈-cycloalkyl, ₄₋₇-heterocycloalkyl, phenyl, monocyclic heteroaryl and C₁-C₃-alkylene-R^{6a}.

8. The compound of claim 5, wherein R⁴ and R⁵, together with the carbons to which they are attached, form a 5-membered heteroaryl ring.

9. The compound of claim 8, wherein the heteroaryl ring is selected from the group consisting of oxazole, thiazole, isoxazole and isothiazole.

10. The compound of claim 1, wherein R¹ is -L¹-Ar¹-Ar²; wherein Ar¹ and Ar² are each independently a phenyl or monocyclic heteroaryl group.

11. The compound of claim 10, wherein Ar¹ is a phenyl group; and Ar² is a 6-membered heteroaryl group.

12. The compound of claim 10, wherein Ar¹ is a 6-membered heteroaryl group; and Ar² is a phenyl group.

13. (canceled)

14. The compound of claim 1, wherein R³ is: 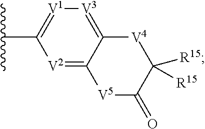
wherein V¹, V² and V³ are each independently selected from the group consisting of N and CR¹⁰;
with the proviso that no more than two of V¹, V² and V³ are N; V⁴ and V⁵ are each independently selected from the group consisting of O, S and NR^{a}; wherein R¹⁰ is independently at each occurrence selected from the group consisting of H, halo, nitro, cyano, NR^{a}R^{a}, NR^{a}S(O)₂R^{a}, NR^{a}C(O)R^{a}, NR^{a}CONR^{a}R^{a}, NR^{a}CO₂R^{a}, OR^{a}, SR^{a}, SOR^{a}, SO₃R^{a}, SO₂R^{a}, SO₂NR^{a}R^{a}, CO₂R^{a}, C(O)R^{a}, CONR^{a}R^{a}, CR^{a}R^{a}NR^{a}R^{a}, CR^{a}R^{a}OR^{a}, C₁-C₄-alkyl, C₂-C₄-alkenyl, C₂-C₄-alkynyl and C₁-C₄-haloalkyl; and R¹⁵ is independently at each occurrence selected from the group consisting of H, fluoro, cyano, CO₂R^{a}, C(O)R^{a}, CONR^{a}R^{a}, C₁-C₄-alkyl, C₂-C₄-alkenyl, C₂-C₄-alkynyl and C₁-C₄-haloalkyl.

15. The compound of claim 1, wherein the compound of formula (I) is selected from the group consisting of:
rel-1-{2-[(3aR*,7aS*)-2-oxo-1-{3-oxo-2H,3H,4H-pyrido[3,2-b][1,4]oxazin-6-yl}-octahydro-[1,3]oxazolo[5,4-c]pyridin-5-yl]ethyl}-7-methoxy-1,2-dihydro-1,8-naphthyridin-2-one;
rel-1-{3-[(3aR*,7aS*)-2-oxo-1-{3-oxo-2H,3H,4H-pyrido[3,2-b][1,4]oxazin-6-yl}-octahydro-[1,3]oxazolo[5,4-c]pyridin-5-yl]propyl}-1,2-dihydroquinolin-2-one;
rel-(3aR*,7aS*)-5-(2-{2-methoxy-7-oxo-7H,8H-pyrido[2,3-d]pyrimidin-8-yl}ethyl)-1-{3-oxo-2H,3H,4H-pyrido[3,2-b][1,4]oxazin-6-yl}-octahydro-[1,3]oxazolo[5,4-c]pyridin-2-one;
rel-1-{2-[(3aR*,7aS*)-2-oxo-1-{3-oxo-2H,3H,4H-pyrido[3,2-b][1,4]oxazin-6-yl}-octahydro-[1,3]oxazolo[5,4-c]pyridin-5-yl]ethyl}-2-oxo-1,2-dihydroquinoline-7-carbonitrile;
rel-1-{2-[(3aR*,7aS*)-2-oxo-1-{3-oxo-2H,3H,4H-pyrido[3,2-b][1,4]oxazin-6-yl}-octahydro-[1,3]oxazolo[5,4-c]pyridin-5-yl]ethyl}-7-methoxy-1,2-dihydro-1,5-naphthyridin-2-one;
rel-1-{3-[(3aR*,7aS*)-2-oxo-1-{3-oxo-2H,3H,4H-pyrido[3,2-b][1,4]oxazin-6-yl}-octahydro-[1,3]oxazolo[5,4-c]pyridin-5-yl]propyl}-2-oxo-1,2-dihydroquinoline-7-carbonitrile;
rel-1-{3-[(3aR*,7aS*)-2-oxo-1-{3-oxo-2H,3H,4H-pyrido[3,2-b][1,4]oxazin-6-yl}-octahydro-[1,3]oxazolo[5,4-c]pyridin-5-yl]propyl}-7-methoxy-1,2-dihydro-1,8-naphthyridin-2-one;
rel-(3aR*,7aR*)-5-(2-{2-methoxy-7-oxo-7H,8H-pyrido[2,3-d]pyrimidin-8-yl}ethyl)-1-{3-oxo-2H,3H,4H-pyrido[3,2-b][1,4]oxazin-6-yl}-octahydro-[1,3]oxazolo[5,4-c]pyridin-2-one;
-rel-1-{3-[(3aR*,7aR*)-2-oxo-1-{3-oxo-2H,3H,4H-pyrido[3,2-b][1,4]oxazin-6-yl}-octahydro-[1,3]oxazolo[5,4-c]pyridin-5-yl]propyl}-7-methoxy-1,2-dihydro-1,8-naphthyridin-2-one; and
rel-1-{3-[(3aR*,7aR*)-2-oxo-1-{3-oxo-2H,3H,4H-pyrido[3,2-b][1,4]oxazin-6-yl}-octahydro-[1,3]oxazolo[5,4-c]pyridin-5-yl]propyl}-2-oxo-1,2-dihydroquinoline-7-carbonitrile.

16-23. (canceled)

24. A pharmaceutical composition comprising a compound of claim 1; and at least one pharmaceutically acceptable excipient.

25. A method of treating a bacterial infection or a mycobacterial infection comprising administering to a patient in need thereof a therapeutic amount of a compound of formula (I), or a pharmaceutically acceptable salt or N-oxide thereof: 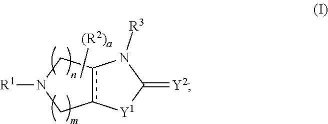
wherein [symbol] is a double bond or a single bond;
Y¹ is independently selected from the group consisting of O and S;
Y² is independently selected from the group consisting of O and S;
R¹ is independently selected from the group consisting of -L¹-Ar¹-Ar² and 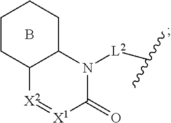
Ar¹ and Ar² are each independently a phenyl or monocyclic heteroaryl group;
-L¹- is —C₁-C₃-alkylene-;
X¹ is independently selected from the group consisting of N and CR⁴; and X² is independently selected from the group consisting of N and CR⁵; or
X¹ and X² together form a 5-membered heteroaryl ring;
-L²- is —C₂-C₃-alkylene-;
Ring B is independently selected from the group consisting of phenyl, monocyclic 6-membered heteroaryl and pyridinone, optionally substituted with a single —Y³—R⁶ group; Y³ is absent or is independently selected from the group consisting of NR⁷, O and S; where Ring B is a pyridinone ring, the nitrogen of the Ring B pyridinone may be attached to the proximal end of a —C₁-C₃-alkylene-group that is attached at its distal end to the group -L²-;
R² is independently at each occurrence selected from the group consisting of halo, nitro, cyano, NR⁸R⁹, NR⁸S(O)₂R⁸, NR⁸CONR⁸R⁸, NR⁸C(O)R⁸, NR⁸CO₂R⁸, OR⁸, SR⁸, SOR⁸, SO₃R⁸, SO₂R⁸, SO₂NR⁸R⁸, CO₂R⁸, C(O)R⁸, CONR⁸R⁸, C₁-C₄-alkyl, C₂-C₄-alkenyl, C₂-C₄-alkynyl, C₁-C₄-haloalkyl and O—C₁-C₄-haloalkyl;
R³ is a bicyclic carbocyclic or a bicyclic heterocyclic ring system in which at least one of the two rings is aryl or heteroaryl;
or R³ is -L³-phenyl; wherein -L³- is selected from the group consisting of —CR⁸═CR⁸— and —C₄-cycloalkyl-;
R⁴ and R⁵ are each independently selected from the group consisting of H, halo, cyano, C₁-C₄-alkyl and O—C₁-C₄-alkyl;
R⁶ is independently selected from the group consisting of H, C₁-C₄-alkyl, C₂-C₄-alkenyl, C₂-C₄-alkynyl, C₁-C₄-haloalkyl, C₃-C₈-cycloalkyl, ₄₋₇-heterocycloalkyl, phenyl, monocyclic heteroaryl and C₁-C₃-alkylene-R^{6a}; wherein R^{6a} is independently selected from the group consisting of C₃-C₈-cycloalkyl, ₄₋₇-heterocycloalkyl, phenyl and monocyclic heteroaryl;
R⁷ is independently selected from the group consisting of H and C₁-C₄-alkyl;
or R⁶ and R⁷ together with the nitrogen to which they are attached form a 4- to 7-membered heterocycloalkyl ring;
R⁸ is independently at each occurrence selected from the group consisting of H and C₁-C₄-alkyl;
R⁹ is independently selected from the group consisting of H, C₁-C₄-alkyl, C₁-C₄-haloalkyl, S(O)₂—C₁-C₄-alkyl and C(O)—C₁-C₄-alkyl;
a is an integer from 0 to 4;
n and m are each an integer selected from the group consisting of 1 and 2; wherein the sum of n and m is 2 or 3;
wherein any of the aforementioned alkyl, alkylene, alkenyl, alkynyl, haloalkyl, cycloalkyl, carbocyclic, heterocyclic, heterocycloalkyl, aryl, phenyl and heteroaryl groups is optionally substituted, where chemically possible, by 1 to 5 substituents which are each independently at each occurrence selected from the group consisting of oxo, ═NR^{a}, ═NOR^{a}, halo, nitro, cyano, NR^{a}R^{a}, NR^{a}S(O)₂R^{a}, NR^{a}C(O)R^{a}, NR^{a}CONR^{a}R^{a}, NR^{a}CO₂R^{a}, OR^{a}, SR^{a}, SOR^{a}, SO₃R^{a}, SO₂R^{a}, SO₂NR^{a}R^{a}, CO₂R^{a}, C(O)R^{a}, CONR^{a}R^{a}, CR^{a}R^{a}NR^{a}R^{a}, CR^{a}R^{a}OR^{a}, C₁-C₄-alkyl, C₂-C₄-alkenyl, C₂-C₄-alkynyl, C₁-C₄-haloalkyl and O—C₁-C₄-haloalkyl; wherein R^{a} is independently at each occurrence selected from the group consisting of H C₁-C₄ alkyl.

26. The method of claim 25, wherein the bacterial infection is caused by a Gram-positive bacteria.

27. The method of claim 25, wherein the bacterial infection is caused by a Gram-negative bacteria.

28. The method of claim 25, wherein the bacterial infection is caused by a bacterial strain that is resistant to at least one approved antibacterial drug.

29. The method of claim 25, wherein the mycobacterial infection is caused by mycobacteria.

30. The method of claim 25, wherein the mycobacterial infection is caused by a mycobacterial strain that is resistant to at least one approved antimycobacterial drug.
